SEQUENCE LISTING (15 sequences)

SEQ ID NO: 1 - protein, 112 aa, Artificial Sequence
  HuAM4G7 VL
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 2 - protein, 121 aa, Artificial Sequence
  HuAM4G7 VH
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 3 - protein, 107 aa, Homo sapiens [sequence omitted]

SEQ ID NO: 4 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 330 aa, Artificial Sequence
  S267E/L328F IgG1 constant chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, Artificial Sequence
  G236D/S267E IgG1 constant chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLDGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 219 aa, Artificial Sequence
  HuAM4G7 light chain (VH-Ck)
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial Sequence
  HuAM4G7 IgG1 heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 451 aa, Artificial Sequence
  HuAM4G7 S267E/L328F heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 16 aa, Artificial Sequence
  LCDR1
RSSKSLQNVNGNTYLY

SEQ ID NO: 11 - protein, 7 aa, Artificial Sequence
  LCDR2
RMSNLNS

SEQ ID NO: 12 - protein, 9 aa, Artificial Sequence
  LCDR3
MQHLEYPIT

SEQ ID NO: 13 - protein, 5 aa, Artificial Sequence
  HCDR1
SYVMH

SEQ ID NO: 14 - protein, 14 aa, Artificial Sequence
  HCDR2
WIGYINPYNDGTKY

SEQ ID NO: 15 - protein, 12 aa, Artificial Sequence
  HCDR3
GTYYYGTRVFDY

(19) United States
(12) Patent Application Publication
(10) Pub. No.: US 2024/0262907 A1
(43) Pub. Date: Aug. 8, 2024

(54) BIOMARKERS, METHODS, AND COMPOSITIONS FOR TREATING AUTOIMMUNE DISEASE INCLUDING SYSTEMIC LUPUS ERYTHEMATOUS (SLE)

(71) Applicant: Xencor, Inc., Pasadena, CA (US)

(72) Inventors: Ying DING, Pasadena, CA (US); John R. DESJARLAIS, Pasadena, CA (US); Bart BURINGTON, Pasadena, CA (US); Raphael CLYNES, Pasadena, CA (US); Debra J. ZACK, Pasadena, CA (US); Paul FOSTER, Pasadena, CA (US); Joel GUTHRIDGE, Pasadena, CA (US)

(73) Assignee: Xencor, Inc., Pasadena, CA (US)

(21) Appl. No.: 18/248,030

(22) PCT Filed: Oct. 6, 2021
(86) PCT No.: PCT/US2021/053790

§ 371 Date: Apr. 5, 2023

Related U.S. Application Data
(60) Provisional application No. 63/088,444, filed on Oct. 6, 2020.
(60) Provisional application No. 63/108,138, filed on Oct. 30, 2020.

(51) Int. Cl. C07K 16/28; A61P 37/06; G01N 33/564

(52) U.S. Cl. CPC C07K 16/2803; A61P 37/06; G01N 33/564; C07K 2317/31; G01N 2800/104; G01N 2800/52

(57) ABSTRACT

The disclosure relates to methods of detecting one or more biomarkers. and/or treating autoimmune diseases such as systemic lupus erythematous (SLE) with an anti-CD19 antibody based on the presence of such biomarkers. The biomarkers can be separate or in combination, and their detection can be used in multiple different methods.

### CROSS-REFERENCE TO RELATED APPLICATIONS

This application claims priority to U.S. Provisional Application No. 63/088,444 (filed Oct. 6, 2020), and U.S. Provisional Application No. 63/108, 138 (filed Oct. 30, 2020), the entire contents of each are incorporated by reference herein.

### SEQUENCE LISTING

The instant application contains a Sequence Listing, which has been filed electronically in ASCII format and is hereby incorporated by reference in its entirety. Said ASCII copy, created Sep. 27, 2021, is named P295266_WO_01_SL.txt and is 23, 152 bytes in size.

### TECHNICAL FIELD

The disclosure relates to biomarkers of systemic lupus erythematous (SLE) treatment efficacy, methods of treating SLE with an anti-CD19 antibody, and compositions that may be useful for such methods.

### BACKGROUND

SLE is a chronic systemic autoimmune disease that may affect multiple organs. The ACR Classification criteria for SLE (Tan et al., 1982) include malar rash, discoid rash, photosensitivity, arthritis, serositis, renal disorder, neurologic disorder, hematologic disorder, immunologic disorder (autoantibodies), and antinuclear antibody (ANA), of which any 4 of the 11 can be present to classify a human subject as having SLE. SLE is typically a disease of young women ages 15-45, with a 10-fold greater incidence in women than in men. There are some ethnic differences, with greater prevalence and severity of disease in persons of African, Hispanic, Asian, and Native American descent. While the risk of mortality has decreased to 5-10% at 10 years, human subjects still die of active disease, infection, cardiovascular causes, and treatment associated effects. Despite improved survival, it is estimated that only 15% of human subjects have good to excellent disease control sustained for 1 year.

SLE is currently incurable. The goals of treatment are to reduce inflammation and damage to the organs and to prevent or reverse disease exacerbations. Therapy is tailored to the organ systems involved and the amount of inflammation, but most of the agents used are non-specific immunosuppressants that are frequently used in combination. For milder forms of SLE involving skin and/or joints, topical or low dose oral steroids, hydroxychloroquine, NSAIDs and/or methotrexate are often the mainstay of therapy. Involvement of other organ systems often warrants higher doses of oral steroids together with agents such as azathioprine, mycophenolate mofetil, or belimumab. Aggressive treatment is warranted when vital organs are involved to prevent organ damage or failure. High dose oral or IV corticosteroids with cyclophosphamide, azathioprine, or mycophenolate mofetil may be used for organ threatening disease in the kidneys, CVS, and hematopoietic systems. Many of the therapeutics are less than optimal therapies particularly for young women because of their long-term safety profiles. For example, long-term corticosteroid use can lead to hypertension, diabetes, osteoporosis, and infection risk, whereas cyclophosphamide may lead to sterility and bladder cancer. Only one new therapy for SLE, belimumab, has been approved in over 50 years. Therefore, there is a need for more targeted agents to control the disease long-term.

In previous Phase 2 SLE trial results for obexelimab (used herein interchangeably as “XmAb5871” and “HuAMAG7”), an anti-CD19 antibody with increased FcyRIlb binding that suppresses B-cell activation, the primary endpoint, which measured loss of baseline response to steroids, was not met.

### SUMMARY

In one aspect, the disclosure is directed to a method of improving therapeutic efficacy for treatment of an autoimmune disease. The expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a subject having SLE is determined. Alternatively, the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127) in subject having SLE is determined. An increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

In another aspect, the disclosure is directed to a method of determining susceptibility to treatment for an autoimmune disease in a human subject in need thereof. The expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject is determined. An increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

In another aspect, the disclosure is directed to a method of selecting one or more human subjects for treating an autoimmune disease or reducing symptoms thereof. The expression of determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more subjects is determined. An increase in expression of the one or more biomarkers indicates that antibody therapy will be effective in the subject.

In another aspect, an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will be efficacious in the subject.

In another aspect, the absence of an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will not be efficacious in the subject.

In another aspect, the absence of an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will be harmful to the subject.

In another aspect, the disclosure is directed to a method of improving therapeutic efficacy for treatment of SLE. The expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a subject having SLE is determined. An increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

In another aspect, the disclosure is directed to a method of determining susceptibility to treatment for SLE in a human subject in need thereof. The expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject. An increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

In another aspect, an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will be efficacious in the subject.

In another aspect, the absence of an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will not be efficacious in the subject.

In another aspect, the absence of an increase of the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject indicates that the human anti-CD19 antibody will be harmful to the subject.

In another aspect, the disclosure is directed to a method of treating SLE or reducing symptoms thereof in a human subject in need thereof. The method includes determining an increased expression level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a blood sample of the human subject. If the expression of the one or more biomarkers is increased, a human anti-CD19 antibody is administered. In certain embodiments, the antibody includes an Fc modification selected from S267E, L328F, and a combination thereof as compared to a parent IgG Fc region, wherein the numbering is according to the EU index as in Kabat.

In another aspect, the disclosure is directed to a method of treating or reducing the symptoms of SLE. A human subject in need thereof is selected by determining the increased expression of biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21. If the expression of the one or more biomarkers is increased, a human anti-CD19 antibody is administered. In certain embodiments, the antibody includes an Fc modification selected from S267E, L328F, and a combination thereof as compared to a parent IgG Fc region, wherein the numbering is according to the EU index as in Kabat.

In another aspect, the disclosure is directed to a method for treating SLE in a human subject in thereof by identifying the subject as having blood tissue expressing an elevated level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21. If the subject is identified as having an expression of the one or more biomarkers is increased, a human anti-CD19 antibody is administered. In certain embodiments, the antibody includes an Fc modification selected from S267E, L328F, and a combination thereof as compared to a parent IgG Fc region, wherein the numbering is according to the EU index as in Kabat.

In another aspect, the one or more biomarkers are selected from CD27, TCF7, CD40LG, FOXP3, CD28, APP, IL-3RA (CD123), MAP1A. In a further aspect, the one or more biomarkers are selected from CD27, TCF7, CD40LG, FOXP3, CD28. In a still further aspect, the one or more biomarkers are selected from TRABD2A, ST6GAL1, ATAD5, ATP13A2, SLC17A9, TBC1D4, MAL, ACY3, DNPH1, CNDP2, CLCN5, CALR, ST3GAL5, and USP21.

In another aspect, the one or more biomarkers is selected from CD27, APP, and a combination thereof. In some variations, the biomarker is CD27. In some variations, the biomarker is APP. In some variations, the biomarker is the combination of CD27 and APP.

In another aspect, if the expression of the one or more biomarkers is not increased, then the antibody is withheld from the subject.

In another aspect, the determining or identifying step includes administering a genotyping test to the blood sample of the subject.

In another aspect, the determining or identifying step comprises administering a proteomic test to the blood sample of the subject.

In another aspect, the blood sample is whole blood. Alternatively, the blood sample is selected from T cells, plasmablasts, and a combination thereof. In another variation, the blood sample can be plasmacytoid dendritic cells.

In another aspect, disclosed is a method of treating systemic lupus erythematous (SLE) in a human subject in need thereof with a therapeutically effective amount of an anti-CD19 antibody. In an embodiment, the anti-CD19 antibody comprises: a light chain comprising a variable region having a CDR1 comprising SEQ ID NO: 10, a CDR2 comprising SEQ ID NO: 11, and a CDR3 comprising SEQ ID NO: 12; and a heavy chain comprising a variable region having a CDR1 comprising SEQ ID NO: 13, a CDR2 comprising SEQ ID NO: 14, and a CDR3 comprising SEQ ID NO: 15, wherein the heavy chain comprises amino acid substitutions in the Fc region S267E and L328F as compared to SEQ ID NO: 4, wherein the numbering is according to the EU index, as in Kabat.

In another aspect, disclosed is a method of treating systemic lupus erythematous (SLE) in a human subject in need thereof with a therapeutically effective amount of an anti-CD19 antibody, wherein the anti-CD19 antibody comprises: a light chain; and a heavy chain comprising an amino acid sequence of SEQ ID NO: 2 and amino acid substitutions in the Fc region S267E and L328F as compared to SEQ ID NO: 4, wherein the numbering is according the EU index, as in Kabat.

In another aspect, disclosed is a method of treating systemic lupus erythematous (SLE) in a human subject in need thereof with a therapeutically effective amount of an anti-CD19 antibody, wherein the anti-CD19 antibody comprises: a light chain comprising an amino acid sequence of SEQ ID NO: 7; and a heavy chain comprising an amino acid sequence of SEQ ID NO: 9.

In another aspect, the therapeutically effective amount of the anti-CD19 antibody of the disclosed method(s) is about 5.0 mg/kg every 14 days for at least 10 doses. In an embodiment, the therapeutically effective amount of the anti-CD19 antibody is about 5.0 mg/kg every 14 days for at least 15 doses. In another embodiment, the therapeutically effective amount of the anti-CD19 antibody is about 5.0 mg/kg every 14 days for at least 16 doses.

In another aspect, the therapeutically effective amount of the anti-CD19 antibody is about 125 mg every 7 days. In an embodiment, the therapeutically effective amount of the anti-CD19 antibody is about 125 mg every 7 days for at least 20 doses. In a further embodiment, the therapeutically effective amount of the anti-CD19 antibody is about 125 mg every 7 days for at least 30 doses. In another embodiment, the therapeutically effective amount of the anti-CD19 antibody is about 125 mg every 7 days for at least 32 doses.

In another aspect, the therapeutically effective amount of the anti-CD19 antibody of the disclosed method(s) is about 250 mg every 14 days. In an embodiment, the therapeutically effective amount of the anti-CD19 antibody is about 250 mg every 14 days for at least 10 doses. In another embodiment, the therapeutically effective amount of the anti-CD19 antibody is about 250 mg every 14 days for at least 15 doses. In a further embodiment, the therapeutically effective amount of the anti-CD19 antibody is about 250 mg every 14 days for at least 16 doses.

In another aspect, the anti-CD19 antibody is provided intravenously.

In another aspect, the anti-CD19 antibody is provided subcutaneously.

In yet another aspect, disclosed is use of a therapeutically effective

amount of an anti-CD19 antibody for treating systemic lupus erythematous (SLE) in a human subject in need thereof having increased expression of biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21. In yet another aspect, disclosed is use of a therapeutically effective amount of an anti-CD19 antibody in the manufacture of a medicament for treating systemic lupus erythematous (SLE) in a human subject in need thereof having increased expression of biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21. In an embodiment, the anti-CD19 antibody comprises: a light chain comprising a variable region having a CDR1 comprising SEQ ID NO: 10, a CDR2 comprising SEQ ID NO: 11, and a CDR3 comprising SEQ ID NO: 12; and a heavy chain comprising a variable region having a CDR1 comprising SEQ ID NO: 13, a CDR2 comprising SEQ ID NO: 14, and a CDR3 comprising SEQ ID NO: 15, wherein the heavy chain comprises amino acid substitutions in the Fc region S267E and L328F as compared to SEQ ID NO: 4, wherein the numbering is according to the EU index, as in Kabat. In another embodiment, the anti-CD19 antibody comprises: a light chain comprising an amino acid sequence of SEQ ID NO: 7; and a heavy chain comprising an amino acid sequence of SEQ ID NO: 9.

The methods described herein, in any combination may comprise selecting subject that are relapsed or relapsed or refractory to rituximab.

### BRIEF DESCRIPTION OF THE FIGURES

The application file contains at least one drawing executed in color. Copies of this patent application publication with color drawing(s) will be provided by the Office upon request and payment of the necessary fee.

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1A depicts the down-regulation of an antigen activated B cell by engagement of immune complexes with the inhibitory Fcy receptor FcyRIIb on the B cell surface.

FIG. 1B depicts the co-ligation of the B cell receptor associated membrane protein CD19 and FcγRIIb by obexelimab, resulting in inhibition of many activation pathways in B cells.

FIG. 2 illustrates the timeline of events for the clinical trial.

FIG. 3 depicts the time to loss of improvement (LOI) through the day 225 planned visit.

FIG. 4 depicts the change in SLEDAI from disease NADIR to end of study (mean difference, 95% CI, at last visit 1.404 p=0.0456).

FIG. 5 lists the amino acid sequences of various variable regions, heavy chain constant regions, CDRs and full-length antibodies of embodiments of the invention.

FIG. 6 depicts a cross-validation analysis of CD27 as a single biomarker predictor of obexelimab efficacy.

FIG. 7A depicts correlation of CD27 expression with T cell genes.

FIG. 7B depicts a Kaplan-Meyer predictiveness of multiple T cell genes, showing that CD40L, FoxP3, CD28, TCF7 biomarkers are predictive of obexelimab efficacy.

FIG. 7C shows genes with highest expression in paces in PBMC RNAseq database.

FIGS. 8A-8D show a cross-validated analyses of A) CD27, B) TCF7, C) FOXP3, and D) CD28 expression as a predictor of treatment effectiveness for antibody and placebo subjects.

FIG. 9A depicts a substantial initial reduction in percent baseline CD86 expression compared to placebo as a function of time after obexelimab administration.

FIG. 9B anti-tetanus IgG (u/ml) shows the inhibition of anti-tetanus response for each of the separate cohorts at Day 21.

FIGS. 10A -10C shows that SLE subjects who have an increased time to LOI upon administration with obexelimab have increased expression of APP, IL-3RA (CD123), and MAP1A, respectively.

FIGS. 11A -11D show response rates as measured by are significantly enriched among cDX+(50%) subjects' four landmark endpoints (A) SRI-4 (B) SRI-6 (C) LLDAS (D) BICLA at 32 weeks for subjects positive with the two biomarker CD27 and APP predictive model.

FIGS. 12A -12D depict the combined two CD27 and APP biomarkers is strongly predictive of LOI in SLE subjects.

FIGS. 13A -13D show pharmacodynamic effects of obexelimab (lower curve) compared with placebo (upper curve) in subjects with SLE who had evaluable baseline whole blood transcriptomic (RNA-seq) data and either completed the study without a flare or experienced a flare.

FIG. 14A depicts the predictability of the 40% biomarker positive group.

FIG. 14B depicts the predictability of the 60% biomarker positive group.

FIGS. 15A -15Q depict cross-validated analyses of A) TRABD2A, B) ST6GAL1, C) ATAD5, D) ATP13A2, E) SLC17A9, F) TBC1D4, G) MAL, H) ACY3, I) DNPH1, J) CNDP2, K) CLCN5, L) CALR, M) ST3GAL5, N) USP21, O) CD40LG, P) FOXP3, and Q) TCF7 expression as a predictor of the effectiveness of antibody-treated and placebo subjects.

FIG. 16A-C show additional two gene, four gene, and 5 gene biomarker combinations that are suitable predictors for obexelimab activity. FIG. 16A shows the 4-gene signature of CD27, TCF7, CCR7, and IL7R. FIG. 16B shows the 2-gene signature of CD27 and TCF7. FIG. 16C shows the 5-gene signature of CD27, TCF7, CCR7, IL7R, and CD28.

### DETAILED DESCRIPTION

This disclosure provides biomarkers of systemic lupus erythematous (SLE) treatment efficacy, methods of treating SLE with an anti-CD19 antibody, and compositions that may be useful for such methods.

### Definitions

Described herein are several definitions. Such definitions are meant to encompass grammatical equivalents.

Unless otherwise required by context, singular terms as used herein and in the claims shall include pluralities and plural terms shall include the singular.

The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements. Also, terms such as “element” or “component” encompass both elements and components comprising one unit and elements and components that comprise more than one subunit unless specifically stated otherwise.

As various changes could be made in the above-described compositions, methods, and kits without departing from the scope of the disclosure, it is intended that all matter contained in the above description and in the examples given below, shall be interpreted as illustrative and not in a limiting sense.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

“Administered” or “administration” includes but is not limited to delivery by an injectable form, such as, for example, an intravenous, intramuscular, intradermal or subcutaneous route or mucosal route, for example, as a nasal spray or aerosol for inhalation or as an ingestible solution, capsule or tablet.

“Antibody” means a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (ν), delta (δ), gamma (γ), sigma (σ), and alpha (α) which encode the IgM, IgD, IgG (IgG1, IgG2, IgG3, and IgG4), IgE, and IgA (IgA1 and IgA2) isotypes, respectively. Antibody herein is meant to include full-length antibodies and antibody fragments and may refer to a natural antibody from any organism, an engineered antibody, or an antibody generated recombinantly for experimental, therapeutic, or other purposes.

“Autoimmune diseases” herein include allogenic islet graft rejection, alopecia areata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgG4-RD, IgM polyneuropathies, immune mediated thrombocytopenia, juvenile arthritis, Kawasaki's disease, lichen plantus, lupus erthematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, Takayasu arteritis, temporal arteritis/giant cell arteritis, thrombotic thrombocytopenia purpura, ulcerative colitis, uveitis, vasculitides such as dermatitis herpetiformis vasculitis, vitiligo, and Wegner's granulomatosis.

“CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. One antibody that is specific for CD19 is the disclosed antibody and is described further below. Some additional antibodies specific for CD19 are described in WO2005012493 (U.S. Pat. No. 7,109,304), WO2010053716 (U.S. Ser. No. 12/266,999) (Immunomedics); WO2007002223 (U.S. Pat. No. 8,097,703) (Medarex); WO2008022152 (U.S. Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (U.S. Ser. No. 12/710,442) (Glenmark Pharmaceuticals), WO2012010562 and WO2012010561 (International Drug Development), WO2011147834 (Roche Glycart), and WO 2012/156455 (Sanofi), which are all incorporated by reference in their entireties. In certain embodiments, these additional antibodies may be used in the instant disclosure.

“CDRs” or “complementarity-determining regions” are the loops in the variable domains of the heavy chain and light chain that form an antigen-binding site. Generally, there are six CDRs total, three each per heavy and light chain, designated VH CDR1, VH CDR2, VH CDR3, VL CDR1, VL CDR2, and VL CDR3.

“Constant region” of an antibody means the region of the antibody that is encoded by one of the light or heavy chain immunoglobulin constant region genes. By “constant light chain” or “light chain constant region” as used herein is meant the region of an antibody encoded by the kappa (CK) or lambda (CA) light chains. The constant light chain typically comprises a single domain, and as defined herein refers to positions 108-214 of CK or CA, wherein numbering is according to the EU index. By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full-length IgG antibodies, the constant heavy chain, as defined herein, refers to the N-terminus of the CH1 domain to the C-terminus of the CH3 domain, thus comprising positions 118-447, wherein numbering is according to the EU index.

“Fc” or “Fc region” means the polypeptide comprising the constant region of an antibody excluding the first constant region immunoglobulin domain and, in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

“Fc gamma receptor” or “FcγR” means any member of the family of proteins that bind the IgG antibody Fc region and are substantially encoded by the FcγR genes. In humans this family includes but is not limited to FcγRI (CD64), including isoforms FcγRla, FcγRIb, and FcγRIc; FcγRII (CD32), including isoforms FcγRIIa (including allotypes H131 and R131), FcγRIIb (including FcγRIlb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

“Modification” means an alteration in the physical, chemical, or sequence properties of a protein, polypeptide, antibody, or immunoglobulin. Modifications described herein include amino acid modifications and glycoform modifications.

“Amino acid modification” means an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution S267E refers to a variant polypeptide, in this case a constant heavy chain variant, in which the serine at position 267 is replaced with glutamic acid. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

“Parent polypeptide,” “parent protein,” “parent immunoglobulin,” “precursor polypeptide,” “precursor protein,” or “precursor immunoglobulin” means an unmodified polypeptide, protein, or immunoglobulin that is subsequently modified to generate a variant, e.g., any polypeptide, protein, or immunoglobulin which serves as a template and/or basis for at least one amino acid modification described herein. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an Fc polypeptide that is modified to generate a variant Fc polypeptide, and by “parent antibody” as used herein is meant an antibody that is modified to generate a variant antibody (e.g., a parent antibody may include, but is not limited to, a protein comprising the constant region of a naturally occurring lg).

“Polypeptide” or “protein” means at least two covalently attached amino acids, which includes proteins, polypeptides, oligopeptides, and peptides.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs to of U.S. Pub. No. 20160244525, hereby incorporated by reference.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penally, etc.) are used. In one embodiment, sequence identity is done using the BLAST algorithm, using default parameter.

The degree of identity between an amino acid sequence of the present disclosure (“disclosure sequence”) and the parental amino acid sequence is calculated as the number of exact matches in an alignment of the two sequences, divided by the length of the “disclosure sequence,” or the length of the parental sequence, whichever is the shortest. The result is expressed in percent identity. In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Position” means a location in the sequence of a protein. Positions may be numbered sequentially, or according to an established format, for example the EU index as in Kabat. For example, position 267 is a position in the human antibody IgG1.

“Residue” means a position in a protein and its associated amino acid identity. For example, Serine 267 (also referred to as Ser267, also referred to as S267) is a residue in the human antibody IgG1.

“Synergy,” “synergism,” or “synergistic” mean more than the expected additive effect of a combination. The “synergy,” “synergism,” or “synergistic” effect of a combination.

“Therapeutically effective amount” of a compound or combination is a dose that produces the effects for which it is administered. The does may cure, alleviate, or partially arrest the clinical manifestations of a given disease or disorder and its complications. The exact dose will depend on the purpose of the treatment as well as the weight and general state of the human subject, and will be ascertainable by one skilled in the art using known techniques. It will be understood that determination of an appropriate dosage may be achieved, using routine experimentation, by constructing a matrix of values and testing different points in the matrix, all of which is within the ordinary skills of a trained physician or clinical scientist.

“Variable region” means the region of an immunoglobulin that comprises one or more Ig domains substantially encoded by any of the VK, VA, and/or VH genes that make up the kappa, lambda, and heavy chain immunoglobulin genetic loci, respectively.

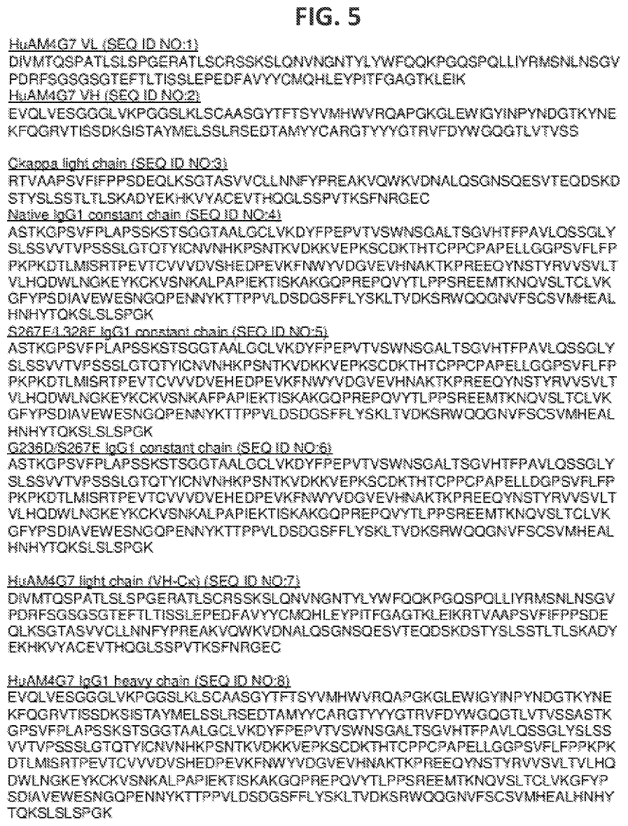

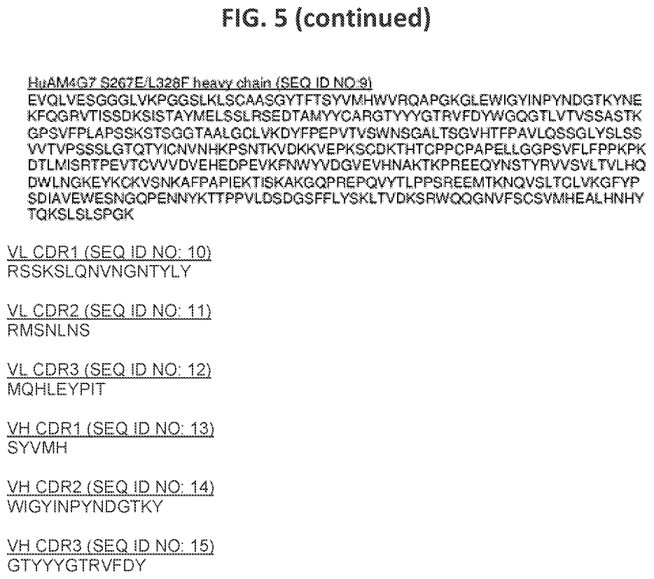

“The disclosed antibody” is an anti-CD19 antibody. The anti-CD19 human antibody includes an Fc modification selected from S267E, L328F, and a combination thereof as compared to a parent IgG Fc region, wherein the numbering is according to the EU index as in Kabat. The disclosed antibody can have CDR, light chain, and heavy chain sequences as described herein. An example amino acid sequence of a variable domains is provided in FIG. 5. The amino acid sequence of the heavy and light chain Fc regions of the disclosed antibody are provided in FIG. 5. The disclosed antibody is described in U.S. Pat. No. 8,063,187, which is incorporated by reference in its entirety. The disclosed antibody has been studied in human clinical trials in systemic lupus erythematous (SLE).

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

The disclosure is directed to a method of improving therapeutic efficacy for treatment of SLE. The expression of one or more biomarkers selected from CD27, TCF7, CD40LG, FOXP3, CD28, APP, IL-3RA (CD123), and MAP1A in a subject having SLE is determined. Increased expression is predictive of effective treatment with a disclosed antibody. The disclosed antibody can be administered to the subject.

### Biomarkers and Identification Thereof

The disclosure is directed to one or more biomarkers that can be used to assess whether a human subject diagnosed with SLE has a larger number of days to loss of improvement (LOI) upon administration of a an anti-CD19 therapeutic antibody with increased Fc binding to FcγIIb. LOI relates to flares that occur in patients. LOI can be a SLEDAI increase ≥4 points or a new BILAG A or B score and physician intent to treat with rescue medication. No loss of LOI refers to maintenance of improvement.

Further, the disclosure is directed to one or more biomarkers that can be used to assess whether a human subject diagnosed with SLE has a lower to an anti-CD19 therapeutic antibody with increased Fc binding to FcγIIb and should not be administered the antibody.

Increased expression level of a biomarker refers to a statistically significant higher expression at Baseline before drug administration that correlates to an increased time to loss of improvement. p values less than 0.05 are considered statistically significant. The statistically significant increase can be based on variables including the number of subjects tested or a specific method of detection or measurement. In circumstances in which multiple biomarkers are detected, p values less than 0.05 for the combination of the biomarkers can be considered statistically significant. The increased expression can be as compared to the expression of a control group, such as the placebo group in a study.

In instances where a longer time to LOI corresponds to higher expression of a biomarker at Baseline, the biomarker alone or in combination with one or more other biomarkers can be detected or measured as part of any method described herein.

Table 1 depicts biomarkers that have higher expression level at Baseline based on an LOI time-to-event endpoint. Cox regression was used to estimate hazard ratios (HR)—the relative likelihood of loss of improvement among obexelimab treated versus Placebo treated subjects at any given point in time. P-values associated with smaller HRs in RNAhigh (cDx+) vs RNAlow (cDx−) were used to rank the predictiveness of candidate genes. Five-fold cross validation and subsampling were used to assess the generalizability of the prediction procedure and robustness of the model.

TABLE 1

| | | Hazard Ratio | Hazard Ratio | |
|---|---|---|---|---|
| | Interaction | Biomarker | Biomarker | |
| Biomarker | p value | Positive | Negative | Cutoff |
| APP | 6.61E−05 | 0.115131869 | 2.065879062 | 24.50317 |
| CD27 | 0.000381893 | 0.147459589 | 1.76595559 | 37.81024 |
| TRABD2A | 0.000948814 | 0.147082432 | 1.626820701 | 44.12328 |
| ST6GAL1 | 0.00168262 | 0.192795103 | 1.389039921 | 124.0642 |
| ATAD5 | 0.00249922 | 0.218080508 | 1.441092263 | 1.298641 |
| ATP13A2 | 0.002777517 | 0.193547303 | 1.315763375 | 10.53048 |
| SLC17A9 | 0.003018473 | 0.199037038 | 1.504572425 | 2.192007 |
| TBC1D4 | 0.003273496 | 0.18963114 | 1.424992809 | 14.64852 |
| MAL | 0.004224301 | 0.20608495 | 1.37106977 | 44.12626 |
| ACY3 | 0.004651756 | 0.242916049 | 1.241810481 | 0.207773 |
| DNPH1 | 0.005937239 | 0.189611029 | 1.298320686 | 12.58014 |
| CNDP2 | 0.007164007 | 0.194293436 | 1.194129953 | 55.52997 |
| CLCN5 | 0.007682673 | 0.257656447 | 1.11816625 | 1.973067 |
| CALR | 0.007707503 | 0.246468741 | 1.223506022 | 111.0891 |
| ST3GAL5 | 0.008794551 | 0.218003642 | 1.195701165 | 24.56635 |
| CD40LG | 0.037267256 | 0.28922067 | 1.163225104 | 12.13703 |
| CD28 | 0.00779928 | 0.309620025 | 1.133954366 | 21.7305 |
| FOXP3 | 0.025462909 | 0.258264408 | 1.053996092 | 3.168203 |
| TCF7 | 0.042302503 | 0.313697068 | 1.082347335 | 198.4939 |
| USP21 | 9.91E−05 | 0.111970154 | 1.851032638 | 11.62798 |

In some variations, after administration of the antibody to a subject, a longer time to LOI corresponds to increased expression of one or more biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21. In some variations, a longer time to LOI corresponds to increased expression of two or more, alternatively three or more, alternatively four or more, alternatively five or more, alternatively six or more, alternatively seven or more, alternatively eight or more, alternatively nine or more, alternatively ten or more, or each biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21.

In some variations, after administration of the antibody to a subject, a longer time to loss of improvement (LOI) corresponds to increased expression of one or more biomarker selected from CD27, TCF7, CD40LG, FOXP3, CD28, APP, IL-3RA (CD123), and MAP1A. In some variations, a longer time to LOI corresponds to increased expression of two or more, alternatively three or more, alternatively four or more, alternatively five or more, alternatively six or more, alternatively seven or more, alternatively eight or more, biomarker selected from CD27, TCF7, CD40LG, FOXP3, CD28, APP, IL-3RA (CD123), and MAP1A.

In some variations, after administration of the antibody to a subject, a longer time to loss of improvement (LOI) corresponds to increased expression of one or more biomarker selected from CD27, TCF7, CD40LG, FOXP3, and CD28. In some variations, after administration of the antibody to a subject, a longer time to loss of improvement (LOI) corresponds to increased expression of two or more, alternatively three or more, alternatively four or more one or more biomarker selected from CD27, TCF7, CD40LG, FOXP3, and CD28. In some variations, after administration of the antibody to a subject, a longer time to loss of improvement (LOI) corresponds to increased expression of the biomarker CD27. In some variations, after administration of the antibody to a subject, a longer time to loss of improvement (LOI) corresponds to increased expression of the biomarker TCF7. In some variations, after administration of the antibody to a subject, a longer time to loss of improvement (LOI) corresponds to increased expression of the biomarker CD40LG. In some variations, after administration of the antibody to a subject, a longer time to loss of improvement (LOI) corresponds to increased expression of the biomarker FOXP3. In some variations, after administration of the antibody to a subject, a longer time to loss of improvement (LOI) corresponds to increased expression of the biomarker CD28. In some variations, after administration of the antibody to a subject, a longer time to loss of improvement (LOI) corresponds to increased expression of the biomarkers CD27, TCF7, CD40LG, FOXP3, and CD28.

In some variations, after administration of the antibody to a subject, a longer time to LOI corresponds to increased expression of one or more biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127). In some variations, a longer time to LOI corresponds to increased expression of two or more, alternatively three or more, alternatively four or more, alternatively five or more, alternatively six or more, alternatively seven or more, alternatively eight or more, alternatively nine or more, alternatively ten or more or each biomarker selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127).

In some variations, after administration of the antibody to a subject, a longer time to loss of improvement (LOI) corresponds to increased expression of one or more biomarker selected from APP, IL-3RA (CD123), and MAP1A. In some variations, after administration of the antibody to a subject, a longer time to loss of improvement (LOI) corresponds to increased expression of the biomarker APP. In some variations, after administration of the antibody to a subject, a longer time to loss of improvement (LOI) corresponds to increased expression of the biomarker IL-3RA. In some variations, after administration of the antibody to a subject, a longer time to loss of improvement (LOI) corresponds to increased expression of the biomarker IL-3RA(CD123). In some variations, after administration of the antibody to a subject, a longer time to loss of improvement (LOI) corresponds to increased expression of the biomarker MAP1A. In some variations, after administration of the antibody to a subject, a longer time to loss of improvement (LOI) corresponds to increased expression of one or more biomarkers selected from CD27, TCF7, CD40LG, FOXP3, and CD28, and one or more biomarkers selected from APP, IL-3RA (CD123), and MAP1A. In some variations, after administration of the antibody to a subject, a longer time to loss of improvement (LOI) corresponds to increased expression of the combination of biomarkers CD27 and APP.

In some variations, after administration of the antibody to a subject, a longer time to LOI corresponds to increased expression of one or more biomarker selected from CD27, TCF7, CD40LG, FOXP3, CD28, APP, IL-3RA (CD123), and MAP1A. In some variations, after administration of the antibody to a subject, a longer time to LOI corresponds to increased expression of one or more biomarker selected from CD27, TCF7, CD40LG, FOXP3, and CD28. In some variations, after administration of the antibody to a subject, a longer time to LOI corresponds to increased expression of the biomarker CD27. In some variations, after administration of the antibody to a subject, a longer time to LOI corresponds to increased expression of the biomarker TCF7. In some variations, after administration of the antibody to a subject, a longer time to LOI corresponds to increased expression of the biomarker CD40LG. In some variations, after administration of the antibody to a subject, a longer time to LOI corresponds to increased expression of the biomarker FOXP3. In some variations, after administration of the antibody to a subject, a longer time to LOI corresponds to increased expression of the biomarker CD28.

In some variations, after administration of the antibody to a subject, a longer time to LOI corresponds to increased expression of one or more biomarker selected from APP, IL-3RA (CD123), and MAP1A. In some variations, after administration of the antibody to a subject, a longer time to LOI corresponds to increased expression of the biomarker APP. In some variations, after administration of the antibody to a subject, a longer time to LOI corresponds to increased expression of the biomarker IL-3RA. In some variations, after administration of the antibody to a subject, a longer time to LOI corresponds to increased expression of the biomarker MAP1A. In some variations, after administration of the antibody to a subject, a longer time to LOI corresponds to increased expression of each of the biomarkers APP, IL-3RA (CD123), and MAP1A.

In some variations, the biomarker is selected from one or more of TRABD2A, ST6GAL1, ATAD5, ATP13A2, SLC17A9, TBC1D4, MAL, ACY3, DNPH1, CNDP2, CLCN5, CALR, ST3GAL5, and USP21. In some variations, after administration of the antibody to a subject, a longer time to LOI corresponds to increased expression of the biomarker TRABD2A. In some variations, after administration of the antibody to a subject, a longer time to LOI corresponds to increased expression of the biomarker ST6GAL1. In some variations, after administration of the antibody to a subject, a longer time to LOI corresponds to increased expression of the biomarker ATAD5. In some variations, after administration of the antibody to a subject, a longer time to LOI corresponds to increased expression of the biomarker ATP13A2. In some variations, after administration of the antibody to a subject, a longer time to LOI corresponds to increased expression of the biomarker SLC17A9. In some variations, after administration of the antibody to a subject, a longer time to LOI corresponds to increased expression of the biomarker TBC1D4. In some variations, after administration of the antibody to a subject, a longer time to LOI corresponds to increased expression of the biomarker MAL. In some variations, after administration of the antibody to a subject, a longer time to LOI corresponds to increased expression of the biomarker ACY3. In some variations, after administration of the antibody to a subject, a longer time to LOI corresponds to increased expression of the biomarker DNPH1. In some variations, after administration of the antibody to a subject, a longer time to LOI corresponds to increased expression of the biomarker CNDP2, CLCN5. In some variations, after administration of the antibody to a subject, a longer time to LOI corresponds to increased expression of the biomarker CALR. In some variations, after administration of the antibody to a subject, a longer time to LOI corresponds to increased expression of the biomarker ST3GAL5. In some variations, after administration of the antibody to a subject, a longer time to LOI corresponds to increased expression of the biomarker USP21.

In some variations, after administration of the antibody to a subject, a longer time to LOI corresponds to increased expression of one or more biomarkers selected from CD27, TCF7, CD40LG, FOXP3, and CD28, and one or more biomarkers selected from APP, IL-3RA (CD123), and MAP1A. In some variations, after administration of the antibody to a subject, a longer time to LOI corresponds to increased expression of the combination of biomarkers CD27 and APP.

In some variations, after administration of the antibody to a subject, a longer time to LOI corresponds to increased expression of one or more of CD27, TCF7, CCR7, IL7R, and CD28.

In alternative variations, after administration of the antibody to a subject, a longer time to LOI corresponds to increased expression of CD27 and TCF7. In other variations, after administration of the antibody to a subject, a longer time to LOI corresponds to increased expression of CD27, TCF7, CCR7, and IL7R. In alternate variations, after administration of the antibody to a subject, a longer time to LOI corresponds to increased expression of CD27, TCF7, CCR7, IL7R, and CD28.

In some variations, determining the expression level of multiple biomarkers can be more robust than determining the expression level of a single biomarker. This is particularly true where the genes associated with the biomarkers are in different cellular pathways. For example, the expression level of one or more biomarkers selected from CD27, TCF7, CD40LG, FOXP3, and CD28 and one or more biomarkers selected from APP, IL-3RA (CD123), and MAP1A biomarkers provide for robust prediction. In some variations, the expression level of three or more, four or more, five or more, six or more, seven or more, eight or more, nine or more, or ten or more biomarkers can be determined.

The antibodies administered herein reduce B cell numbers in treated SLE subjects. The antibodies further reduce RNA markers of circulating total and activated B cells as well as plasma cells. The gene expression analysis points to additional involvement of both pDCs and Tcells to obexelimab responses. APP is highly expressed in pDCs. CD27 expression was highly correlative with other canonical resting T and Tscm signature genes (TCF7, CD28, CCR7, IL-7R) in SLE subjects.

The biomarkers referred to herein can be found by, for example, NCBI accession number, GI number, and the like.

The biomarker expression level can be determined from whole blood. Alternatively, the expression can be measured from different cell types that are separated, isolated, and/or increased in concentration in a sample. In some variations, the expression of biomarkers can be determined from samples of whole blood. In some additional variations, samples can include isolated peripheral blood mononuclear cells (PBMCs), or isolated T cells, such as isolated CD8+ cells or isolated CD4+ T cells, as employed in the methods of the prior art. The sample may be subjected to processing following sample collection, such as cell lysis and/or addition of one or more enzyme inhibitors to inhibit RNA degradation in the whole blood sample. In some variations, the expression of biomarkers can be determined from T cells, plasmablasts, and combinations thereof. In some variations, the expression of biomarkers can be determined from dendritic cells, such as plasmacytoid dendritic cells.

Another aspect of the disclosure is a method of treating SLE in an individual in need thereof by administering a therapeutically effective amount of an antibody specific for CD19. In certain embodiments, the individual expresses one or more of the biomarkers disclosed herein.

Yet another aspect of the disclosure is use of a therapeutically effective amount of an anti-CD19 antibody for treating systemic lupus erythematous (SLE) in a human subject having increased expression of any of the biomarkers disclosed herein. In yet another aspect of the disclosure is use of a therapeutically effective amount of an anti-CD19 antibody in the manufacture of a medicament for treating systemic lupus erythematous (SLE) in a human subject having increased expression of any of the biomarkers disclosed herein.

As described further in the examples below, it has been surprisingly found that determining an increased expression level of one or more biomarkers disclosed herein provides increased efficacy of one of the disclosed antibody in treating SLE. In some variations, those human subjects with increased expression of one or more biomarkers had an extended time to loss of improvement (LOI). In some variations, those human subjects with increased expression of one or more biomarkers had did not experience loss of improvement as frequently as human subjects without increased expression of one or more biomarkers. In some additional variations, human subjects that did not show an increase of expression of one or more biomarkers showed a reduced time to LOI.

There are many suitable methods that may be used to determine a biomarker expression level.

For example, the level of expression of one or more biomarkers may be compared to a collection of data obtained from a group. The comparison may use a linear regression model. In another example, the level of biomarker expression can be compared with a threshold level for each gene in question. A suitable threshold level for a gene can be determined, for example, using qPCR expression data and machine learning methods (such as logistic regression, support vector machines, or decision tree-based methods) to establish an optimal expression threshold that allows maximal separation of subjects.

Alternatively, the median expression level of one or more biomarkers may be used as a control value, wherein the group consisted of subjects, preferably at least 100, at least 50, or at least 10 subjects. In this case, an above median expression level of biomarkers may indicate that the antibody described herein should be administered, to a subject. If the expression level of one or more biomarkers is below a median expression level of biomarkers, which can indicate that the antibody described herein should not be administered to the subject. As a further alternative, the median expression level of each of the genes in question in samples obtained from a group of subjects, such as at least 100, at least 50, or at least 10 subjects, may be used as a control, wherein the group consisted of subjects.

The level of expression of biomarkers may be determined by any convenient means and many suitable techniques are known in the art. For example, suitable techniques include: real time quantitative PCR (RT-qPCR), digital PCR, microarray analysis, whole transcriptome shotgun sequencing (RNA-SEQ), RNA-Seq by Expectation-Maximization (RSEM), and direct multiplexed gene expression analysis. A method of the invention may therefore comprise bringing a whole blood sample obtained from an subject into contact with a reagent suitable for determining biomarker expression levels e.g. a reagent or reagents suitable for determining the expression level of two or more of said genes using RT-qPCR, digital PCR, microarray analysis, whole transcriptome shotgun sequencing, direct multiplexed gene expression analysis, ELISA, protein chips, flow cytometry, mass spectrometry, or Western blotting. For example, the reagent may be a pair or pairs of nucleic acid primers, suitable for determining the expression level of one or more of said genes using RT-qPCR, digital PCR, or whole transcriptome shotgun sequencing. Alternatively, the reagent may be an antibody suitable for determining the expression level of said one or more genes using ELISA or Western blotting. Preferably, the level of expression of said genes is determined using RT-qPCR, digital PCR, microarray analysis, whole transcriptome shotgun sequencing, or direct multiplexed gene expression analysis. Most preferably, the level of expression of said genes is determined using RT-qPCR.

RT-qPCR allows amplification and simultaneous quantification of a target DNA molecule. To analyze gene expression levels using RT-qPCR, the total mRNA of a whole blood sample may first be isolated and reverse transcribed into cDNA using reverse transcriptase. For example, mRNA levels can be determined using e.g. Taqman Gene Expression Assays (Applied Biosystems) on an ABI PRISM 7900HT instrument according to the manufacturer's instructions. Transcript abundance can then be calculated by comparison to a standard curve.

Digital PCR can also be used to detect biomarkers. Digital PCR works by partitioning a sample of DNA or cDNA into many subject, parallel PCR reactions; some of these reactions contain the target molecule (positive) while others do not (negative). A single molecule can be amplified a million-fold or more. During amplification, TaqMan® chemistry with dye-labeled probes is used to detect sequence-specific targets. When no target sequence is present, no signal accumulates. Following PCR analysis, the fraction of negative reactions is used to generate an absolute count of the number of target molecules in the sample, without the need for standards or endogenous controls. The use of a nanofluidic chip provides a convenient and straightforward mechanism to run thousands of PCR reactions in parallel. Each well is loaded with a mixture of sample, master mix, and TaqMan® Assay reagents, and analyzed to detect the presence (positive) or absence (negative) of an endpoint signal. To account for wells that may have received more than one molecule of the target sequence, a correction factor is applied using the Poisson model.

RNA-SEQ uses next-generation sequencing (NGS) for the detection and quantification of RNA in a biological sample at a given moment in time. An RNA library is prepared, transcribed, fragmented, sequenced, reassembled and the sequence or sequences of interest quantified.

NanoString technology uses unique color-coded molecular barcodes that can hybridize directly to many different types of target nucleic acid molecules, and offers a cost-effective way to analyze the expression levels of up to 800 genes simultaneously, with sensitivity comparable to qPCR.

Flow-FISH for RNA employs flow cytometry to determine the abundance of a target mRNA within a sample using fluorescently-tagged RNA oligos. This technique is described, for example, in Porichis et al., Nat Comm (2014) 5:5641. The advantage of this technique is that it can be used without the need to separate the cells present in a sample.

Microarrays allow gene expression in two samples to be compared. Total RNA is first isolated from, e.g. PBMCs or whole blood using, for example, Trizol or an RNeasy mini kit (Qiagen). The isolated total RNA is then reverse transcribed into double-stranded cDNA using reverse transcriptase and polyT primers and labelled using e.g. Cγ3- or Cγ5-dCTP. Appropriate Cγ3- and Cγ5-labelled samples are then pooled and hybridized to custom spotted oligonucleotide microarrays comprised of probes representing suitable genes and control features, such as the microarray described in (Willcocks et al., J Exp Med205, 1573-82, 2008). Samples may be hybridized in duplicate, using a dye-swap strategy, against a common reference RNA derived from pooled PBMC or whole blood samples. Following hybridization, arrays are washed and scanned on e.g. an Agilent G2565B scanner. Suitable alternatives to the steps described above are well known in the art and would be apparent to the skilled person. The raw microarray data obtained can then be analyzed using suitable methods to determine the relative expression of any of genes CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, CCR7, IL7R, and USP21.

Enzyme-linked immunosorbent assays (ELISAs) allow the relative amounts of proteins present in a sample to be detected. The sample is first immobilized on a solid support, such as a polystyrene microtiter plate, either directly or via an antibody specific for the protein of interest. After immobilization, the antigen is detected using an antibody specific for the target protein. Either the primary antibody used to detect the target protein may be labelled to allow detection, or the primary antibody can be detected using a suitably labelled secondary antibody. For example, the antibody may be labelled by conjugating the antibody to a reporter enzyme. In this case, the plate developed by adding a suitable enzymatic substrate to produce a visible signal. The intensity of the signal is dependent on the amount of target protein present in the sample.

Protein chips, also referred to as protein arrays or protein microarrays, allow the relative amounts of proteins present in a sample to be detected. Different capture molecules may be affixed to the chip. Examples include antibodies, antigens, enzymatic substrates, nucleotides, and other proteins. Protein chips can also contain molecules that bind to a range of proteins. Protein chips are well known in the art and many different protein chips are commercially available.

Western blotting also allows the relative amounts of proteins present in a sample to be determined. The proteins present in a sample are first separated using gel electrophoresis. The proteins are then transferred to a membrane, e.g. a nitrocellulose or PVDF membrane, and detected using monoclonal or polyclonal antibodies specific to the target protein. Many different antibodies are commercially available and methods for making antibodies to a given target protein are also well established in the art. To allow detection, the antibodies specific for the protein(s) of interest, or suitable secondary antibodies, may, for example, be linked to a reporter enzyme, which drives a colorimetric reaction and produces a color when exposed to an appropriate substrate. Other reporter enzymes include horseradish peroxidase, which produces chemiluminescence when provided with an appropriate substrate. Antibodies may also be labelled with suitable radioactive or fluorescent labels. Depending on the label used, protein levels may be determined using densitometry, spectrophotometry, photographic film, X-ray film, or a photosensor.

Flow cytometry allows the relative amounts of proteins present in e.g. a PBMC or whole blood sample obtained from a subject to be determined. Flow cytometry can also be used to detect or measure the level of expression of a protein of interest on the surface of cells. Detection of proteins and cells using flow cytometry normally involves first attaching a fluorescent label to the protein or cell of interest. The fluorescent label may for example be a fluorescently-labelled antibody specific for the protein or cell of interest. Many different antibodies are commercially available and methods for making antibodies specific for a protein of interest are also well established in the art.

Mass spectrometry, e.g. matrix-assisted laser desorption/ionization (MALDI) mass spectrometry, allows the identification of proteins present in a sample obtained from a subject using e.g. peptide mass finger printing. Prior to mass spectrometry the proteins present in the sample may be isolated using gel electrophoresis, e.g. SDS-PAGE, size exclusion chromatography, or two-dimensional gel electrophoresis.

### Kits

Also disclosed is a kit for use in determining one or more biomarkers. The kit may include reagents for establishing the biomarker(s) expression levels. The kit may include reagents for establishing the expression level of three or more, four or more, five or more, six or more, seven or more, eight or more, nine or more, or ten or more biomarkers. For example, the reagents may be reagents suitable for establishing the expression of the genes in question using any technique described herein, such as RT-qPCR, digital PCR, microarray analysis, whole transcriptome shotgun sequencing, or direct multiplexed gene expression analysis. For example, the kit may comprise primers suitable for establishing the level of expression of the genes in question using e.g. RT-qPCR, digital PCR, whole transcriptome shotgun sequencing, or direct multiplexed gene expression analysis. The design of suitable primers is routine and well within the capabilities of the skilled person. A kit for direct multiplexed gene expression analysis may in addition, or alternatively, include fluorescent probes for establishing the level of expression of the genes in question. In addition to detection reagents, the kit may also include RNA extraction reagents and/or reagents for reverse transcription of RNA into cDNA.

A kit may also include one or more articles and/or reagents for performance of the method, such as buffer solutions, and/or means for obtaining the test sample itself, e.g. means for obtaining and/or isolating a sample and sample handling containers (such components generally being sterile). The kit may include instructions for use of the kit in a method for assessing whether to administer an antibody described herein to a subject.

### Examples of Anti-CD19 Antibodies

In various aspects, the anti-CD19 antibody has the amino modification S267E in the Fc region, wherein the numbering is according to the EU index, as in Kabat. In various aspects, the anti-CD19 antibody has the amino modification L328F in the Fc region. In various aspects, the anti-CD19 antibody has the amino modifications S267E and L328F in the Fc region, wherein the numbering is according to the EU index, as in Kabat.

In an embodiment, the anti-CD19 antibody comprises an antibody that binds to the same epitope as an antibody comprising: a light chain comprising a variable region having a CDR1 comprising SEQ ID NO: 10, a CDR2 comprising SEQ ID NO: 11, and a CDR3 comprising SEQ ID NO: 12; and a heavy chain comprising a variable region having a CDR1 comprising SEQ ID NO: 13, a CDR2 comprising SEQ ID NO: 14, and a CDR3 comprising SEQ ID NO: 15, wherein the heavy chain comprises amino acid substitutions in the Fc region S267E and L328F as compared to SEQ ID NO: 4, wherein the numbering is according to the EU index, as in Kabat.

In an embodiment, the anti-CD19 antibody comprises: a light chain comprising a variable region having a CDR1 comprising SEQ ID NO: 10, a CDR2 comprising SEQ ID NO: 11, and a CDR3 comprising SEQ ID NO: 12; and a heavy chain comprising a variable region having a CDR1 comprising SEQ ID NO: 13, a CDR2 comprising SEQ ID NO: 14, and a CDR3 comprising SEQ ID NO: 15, wherein the heavy chain comprises amino acid substitutions in the Fc region S267E and L328F as compared to SEQ ID NO: 4, wherein the numbering is according to the EU index, as in Kabat.

In an embodiment, the anti-CD19 antibody comprises: a light chain; and a heavy chain comprising an amino acid sequence of SEQ ID NO: 2 and amino acid substitutions in the Fc region S267E and L328F as compared to SEQ ID NO: 4, wherein the numbering is according the EU index, as in Kabat.

In an embodiment, the anti-CD19 antibody comprises: a light chain comprising an amino acid sequence of SEQ ID NO: 7; and a heavy chain comprising an amino acid sequence of SEQ ID NO: 9.

In an embodiment, the anti-CD19 antibody comprises: a light chain comprising an amino acid sequence having at least 90%, alternatively at least 95%, alternatively at least 96%, alternatively at least 97%, alternatively at least 98%, alternatively at least 99% sequence identity to the amino acid sequence of SEQ ID NO: 7; and a heavy chain comprising an amino acid sequence having at least 90%, alternatively at least 95%, alternatively at least 96%, alternatively at least 97%, alternatively at least 98%, alternatively at least 99% sequence identity to the amino acid sequence of SEQ ID NO: 9.

### Combinations, Pharmaceuticals, and Pharmaceutical Compositions

The disclosure also relates to combinations, pharmaceuticals, and pharmaceutical compositions containing the described combinations.

The terms “in combination with” and “co-administration” are not limited to the administration of agents at exactly the same time. Instead, it is meant that the anti-CD19 antibody disclosed herein, and another molecule are administered in a sequence and within a time interval such that they may act together to provide a benefit that is increased versus treatment with only the anti-CD19 antibody. In some embodiments, the two components are administered at a time where both components (drugs) are active in the human subject at the same time. Such molecules are suitably present in combination in amounts that are effective for the purpose intended. The skilled medical practitioner can determine empirically, or by considering the pharmacokinetics and modes of action of the agents, the appropriate dose, or doses of each therapeutic agent, as well as the appropriate timings and methods of administration.

In some embodiments, a pharmaceutical composition is provided. Pharmaceutical compositions are contemplated wherein the antibody specific for CD19. Formulations of the anti-CD19 antibody disclosed herein are prepared for storage by mixing said anti-CD19 antibody having the desired degree of purity with optional pharmaceutically acceptable carriers, excipients, or stabilizers (Remington's Pharmaceutical Sciences 16th edition, Osol, A. Ed., 1980, incorporated entirely by reference), in the form of lyophilized formulations or aqueous solutions. The formulations to be used for in vivo administration may be sterile. This is readily accomplished by filtration through sterile filtration membranes or other methods.

The antibodies specific to CD19 disclosed herein may also be formulated as immunoliposomes. A liposome is a small vesicle comprising various types of lipids, phospholipids and/or surfactant that is useful for delivery of a therapeutic agent to a mammal. Liposomes containing the anti-CD19 antibody are prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731, all incorporated entirely by reference. Liposomes with enhanced circulation time are disclosed in U.S. Pat. No. 5,013,556, incorporated entirely by reference. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484, incorporated entirely by reference).

The anti-CD19 antibody and other therapeutically active agents may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

In an embodiment, the antibodies specific to CD19 disclosed herein are formulated for intravenous (IV) administration or subcutaneous (SC) administration. Generally, a formulation for IV or SC administration comprises the anti-CD19 antibody, one or more buffers, one or more tonicity modifiers, one or more solvents, and one or more surfactants.

In embodiments, an IV formulation comprises the anti-CD19 antibody in an amount from about 1 mg to about 50 mg per mL. The amount of anti-CD19 antibody can be about 1 mg to about 500 mg per mL or about 1 mg to about 100 mg per mL or about 1 mg to about 50 mg per mL.

In an embodiment, a SC formulation comprises the anti-CD19 antibody in an amount from about 100 mg to about 250 mg per mL. The amount of anti-CD19 antibody can be about 1 mg to about 500 mg per mL or about 50 mg to about 250 mg per mL or about 100 mg to about 250 mg per mL. In some embodiments, the amount of anti-CD19 antibody is about 125 mg per mL. In some variations, the anti-CD19 antibody can be administered in an amount between 200 mg and 300 mg every 14 days. In some variations, the anti-CD19 antibody can be administered in 250 mg SC every 14 days. In some variations, the anti-CD19 antibody can be administered in an amount between 100 mg and 150 mg SC every 14 days. In some variations, the anti-CD19 antibody can be administered in 120 mg SC every 14 days.

In order to treat a human subject, a therapeutically effective dose of the anti-CD19 antibody disclosed herein may be administered. The exact dose will depend on the purpose of the treatment and will be ascertainable by one skilled in the art using known techniques. Dosages may range from 0.0001 to 100 mg/kg of body weight or greater, for example 0.1, 1, 5, 10, or 50 mg/kg of body weight. In one embodiment, dosages range from about 1 to about 10 mg/kg. In another embodiment, the dosage is about 5 mg/kg.

In some embodiments, antibodies used to treat SLE can be administered at doses of greater than or equal to 0.2 mg/kg. For example, antibodies used to treat SLE can be administered at doses of greater than or equal to 0.1 mg/kg, greater than or equal to 0.5 mg/kg, greater than or equal to 1 mg/kg, greater than or equal to 2 mg/kg, greater than or equal to 5 mg/kg, greater than or equal to 10 mg/kg, greater than or equal to 15 mg/kg, greater than or equal to 20 mg/kg, or greater than or equal to 25 mg/kg.

Alternatively, antibodies used to treat SLE can be administered at doses of greater than or equal to 25 mg/kg, greater than or equal to 50 mg/kg, greater than or equal to 75 mg/kg, greater than or equal to 100 mg/kg, greater than or equal to 125 mg/kg, greater than or equal to 150 mg/kg, greater than or equal to 175 mg/kg, or greater than or equal to 200 mg/kg. In other embodiments, antibodies used to treat SLE can be administered at doses of about 0.2 mg/kg. For example, antibodies used to treat SLE can be administered at doses of about 0.1 mg/kg, about 0.5 mg/kg, about 1 mg/kg, about 2 mg/kg, about 5 mg/kg, about 10 mg/kg, about 15 mg/kg, about 20 mg/kg, or about 25 mg/kg. Alternatively, antibodies used to treat SLE can be administered at doses of about 25 mg/kg, about 50 mg/kg, about 75 mg/kg, about 100 mg/kg, about 125 mg/kg, about 150 mg/kg, about 175 mg/kg, or about 200 mg/kg.

In some embodiments, the anti-CD19 antibody is administered at a dose of about 125 mg. In other embodiments, the anti-CD19 antibody is administered at a dose of about 250 mg.

In some embodiments, only a single dose of the anti-CD19 antibody is used. In other embodiments, multiple doses of the anti-CD19 antibody are administered. The elapsed time between administrations may be less than 1 hour, about 1 hour, about 1-2 hours, about 2-3 hours, about 3-4 hours, about 6 hours, about 12 hours, about 24 hours, about 48 hours, about 2-4 days, about 4-6 days, about 7 days, about 14 days, or more than 14 days. In certain embodiments, the anti-CD19 antibody is administered every 14 days. In some embodiments, the anti-CD19 antibody is administered every 7 days.

In certain embodiments, 125 mg of the anti-CD19 antibody is administered every 7 days. In other embodiments, 250 mg of the anti-CD19 antibody is administered every 14 days.

In some embodiments, the anti-CD19 antibody is administered for at least 1 dose. In other embodiments, the anti-CD19 antibody is administered for at least 2 doses. In other embodiments, the anti-CD19 antibody is administered for at least 3 doses. In other embodiments, the anti-CD19 antibody is administered for at least 4 doses. In other embodiments, the anti-CD19 antibody is administered for at least 5 doses. In other embodiments, the anti-CD19 antibody is administered for at least 6 doses. In other embodiments, the anti-CD19 antibody is administered for at least 7 doses. In other embodiments, the anti-CD19 antibody is administered for at least 8 doses. In other embodiments, the anti-CD19 antibody is administered for at least 9 doses. In other embodiments, the anti-CD19 antibody is administered for at least 10 doses. In other embodiments, the anti-CD19 antibody is administered for at least 11 doses. In other embodiments, the anti-CD19 antibody is administered for at least 12 doses. In other embodiments, the anti-CD19 antibody is administered for at least 13 doses. In other embodiments, the anti-CD19 antibody is administered for at least 15 doses. In other embodiments, the anti-CD19 antibody is administered for at least 16 doses. In other embodiments, the anti-CD19 antibody is administered for at least 17 doses. In other embodiments, the anti-CD19 antibody is administered for at least 18 doses. In other embodiments, the anti-CD19 antibody is administered for at least 19 doses. In other embodiments, the anti-CD19 antibody is administered for at least 20 doses. In other embodiments, the anti-CD19 antibody is administered for at least 25 doses. In other embodiments, the anti-CD19 antibody is administered for at least 30 doses. In other embodiments, the anti-CD19 antibody is administered for at least 35 doses. In other embodiments, the anti-CD19 antibody is administered for at least 40 doses. In other embodiments, the anti-CD19 antibody is administered for at least 45 doses. In other embodiments, the anti-CD19 antibody is administered for at least 50 doses. In other embodiments, the anti-CD19 antibody is administered for at least 55 doses. In other embodiments, the anti-CD19 antibody is administered for at least 60 doses. In other embodiments, the anti-CD19 antibody is administered for greater than 2 doses. In one particular embodiment, the anti-CD19 antibody is administered every 14 days for a total of 16 doses. In another particular embodiment, the anti-CD19 antibody is administered every 14 days for a total of 32 doses.

In some instances, any of the methods described herein can be performed on a subject who is refractory to an immunosuppressive biologic therapy (e.g. rituximab, bortezomib). Subjects that are refractory to the administered immunosuppressive biologic therapy do not respond to a given immunosuppressive biologic, such as rituximab or bortezomib, or exhibit a therapeutic response and then re-developed symptoms of the disease. In some instances, the autoimmune disease (e.g., SLE, rheumatoid arthritis) can be treated by administering the antibody described herein to a subject who is refractory to rituximab.

In some instances, any of the methods described herein can be performed on a subject who is to subject has relapsed following treatment with an immunosuppressive biologic. A relapsed subject has responded to treatment with an immunosuppressive biologic, but re-developed symptoms of the disease. In some instances, the immunosuppressive biologic may be rituximab. In some instances, the immunosuppressive biologic may be bortezomib.

### EXAMPLES

The following examples are included to demonstrate embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1

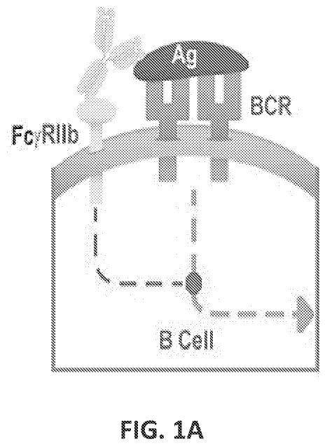

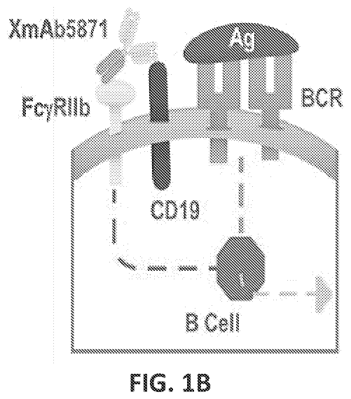

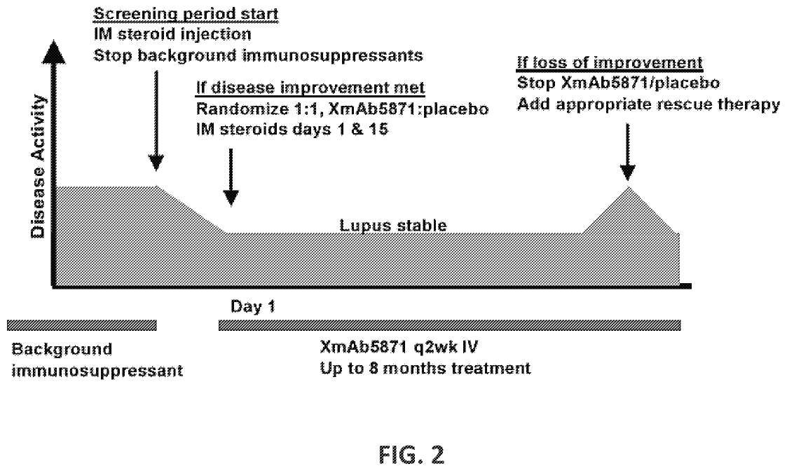

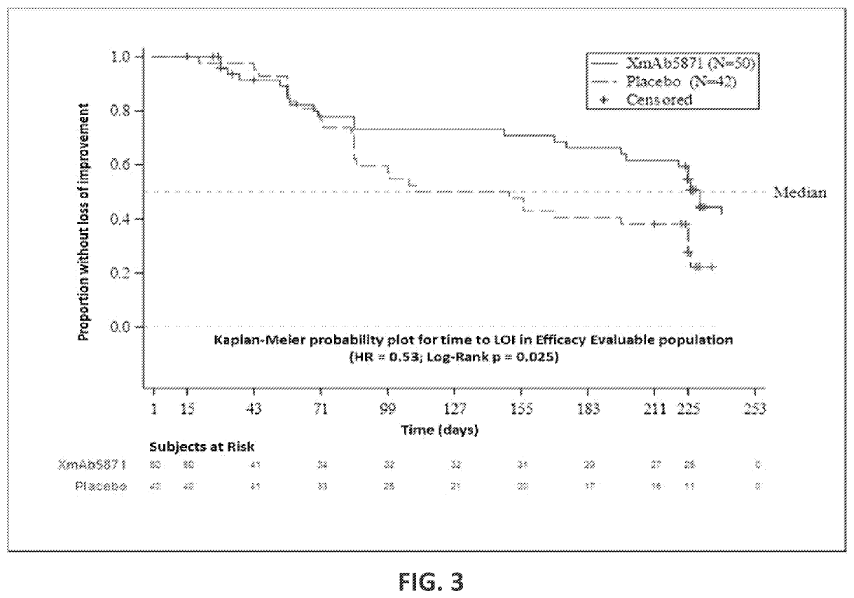

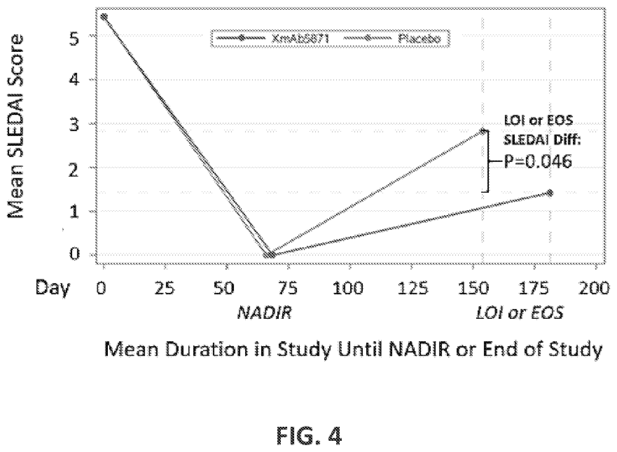

The effects of obexelimab in system lupus erythematosus (SLE) were studied in a phase 2 double-blind, randomized, placebo-controlled study. The down-regulation of an antigen activated B cell by engagement of immune complexes with the inhibitory Fcγ receptor FcγRIIb on the B cell surface is shown in FIG. 1B. FIG. 1B shows a diagram of the mechanism of action of obexelimab, specifically the co-ligation of the B cell receptor associated membrane protein CD19 and FcγRIIb by obexelimab, resulting in inhibition of many activation pathways in B cells. The timeline of the events for the clinical trial are shown in FIG. 2. FIG. 3 and FIG. 4 show the results of the clinical trial, respectively. FIG. 3 depicts the time to loss of improvement (LOI) through the day 225 planned visit. FIG. 4 depicts the change in SLEDAI from disease NADIR to end of study (mean difference, 95% CI, at last visit 1.404 p=0.0456).

Following administration of obexelimab, expression of individual genes and gene pathway scores were evaluated for 68 patients (“patient” is used interchangeably with “subject”) who either completed the study or terminated early for loss of response using a five-fold cross validation framework. Expression of genes was evaluated for both baseline (i.e. before administration of obexelimab) and after treatment. Predictive analysis was based on the baseline/pre-treatment expression, while pharmacodynamic changes were using on treatment samples. The relevance of each gene to a potential biomarker predictive model was measured by the degree to which high or low expression identified a patient subgroup (cDx+) with greater reduction in risk of flare by obexelimab than in cDX− patients.

68 RNAseq whole blood baseline samples were used for predictive biomarker detection. Candidate genes were pre-screened based on 22 immune modules from the AMP phase 1 lupus scRNAseq data. (Arazi A, et al. The immune cell landscape in kidneys of patients with lupus nephritis [published correction appears in Nat Immunol. Nat Immunol. 2019;20(7):902-914.]) Predictive models were based on a Loss of Improvement (LOI) time-to-event endpoint. Cox regression was used to estimate hazard ratios (HR)—the relative likelihood of loss of improvement among obexelimab treated versus Placebo treated subjects at any given point in time. p-values associated with smaller HRs in RNAhigh (cDx+) vs RNAlow (cDx−) were used to rank the predictiveness of candidate genes. Five-fold cross validation and subsampling were used to assess the generalizability of the prediction procedure and robustness of the model.

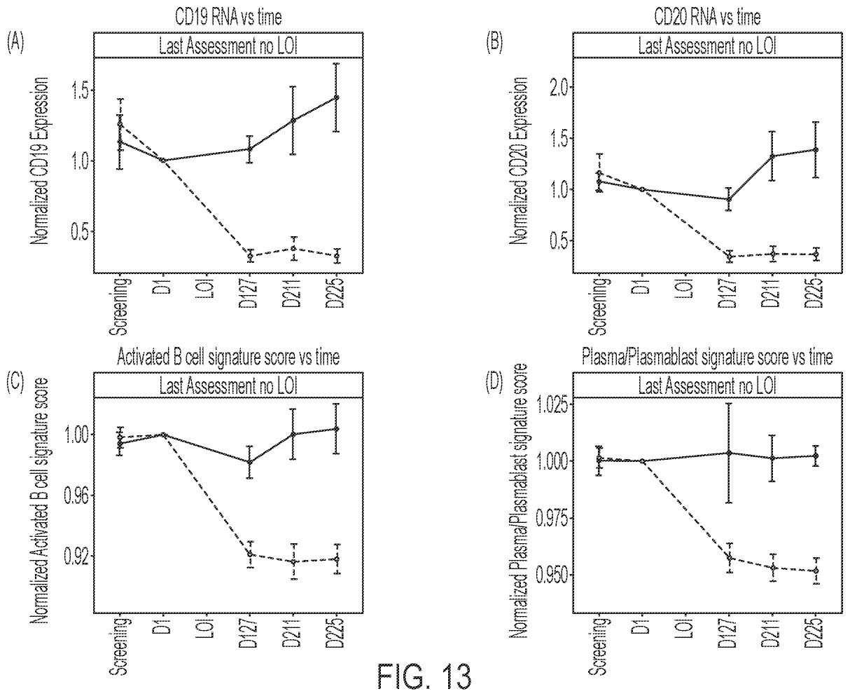

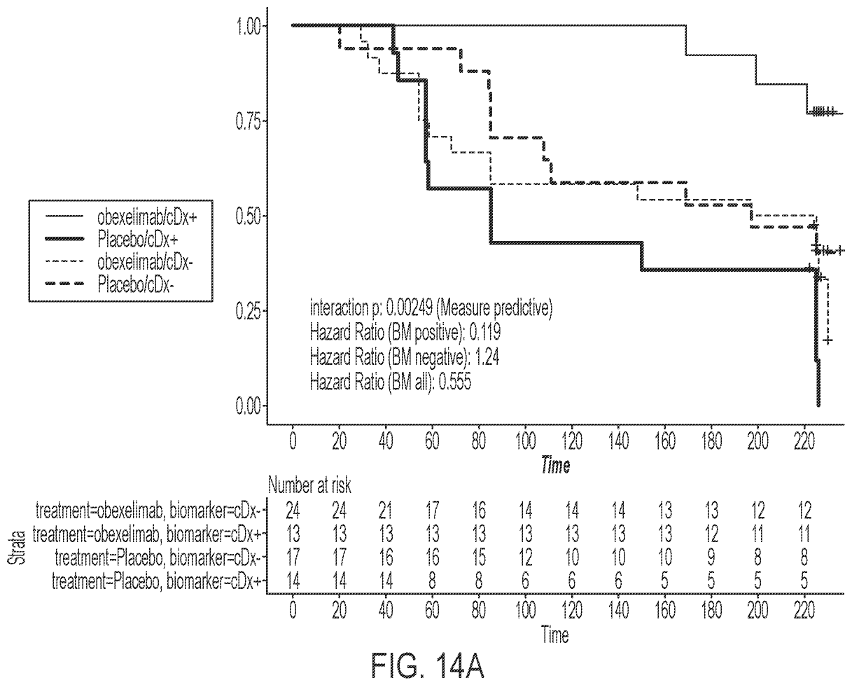

Obexelimab treatment was associated with reduction of B-cell genes and gene-sets reflective of activated B-cells and plasma cells/plasmablasts (FIG. 13). FIG. 13A depicts B-cell marker gene CD19 gene expression over time. FIG. 13B depicts B-cell marker gene CD20 (transcript MS4A1) gene expression over time. FIG. 13C depicts activated B-cell signature score over time. FIG. 13D depicts plasma/plasmablast signature score over time.

FIGS. 13A-13D show pharmacodynamic effects of obexelimab (lower curve) compared with placebo (upper curve) in subjects with SLE who had evaluable baseline whole blood transcriptomic (RNA-seq) data and either completed the study without a flare or experienced a flare on study. FIG. 13A shows normalized CD19 expression over time. FIG. 13B shows normalized CD20 expression up to 225 days. FIG. 13C shows the activated B cell signature score up to 225 days. The RNA expression and signature scores are fold-change relative to baseline. Signature scores are based on immune cell signatures (described in Arazi, A. et al. Nat. Immunol. 2019. 20;902-914, incorporated by reference in its entirety) and generated by the Singscore method (Foroutan M, et al., BMC Bioinformatics, 2018 19; 404, incorporated by reference in its entirety.)

The data show that whole blood RNAseq confirms treatment-related reductions in total and activated B cells and plasmablasts.

### Example 2

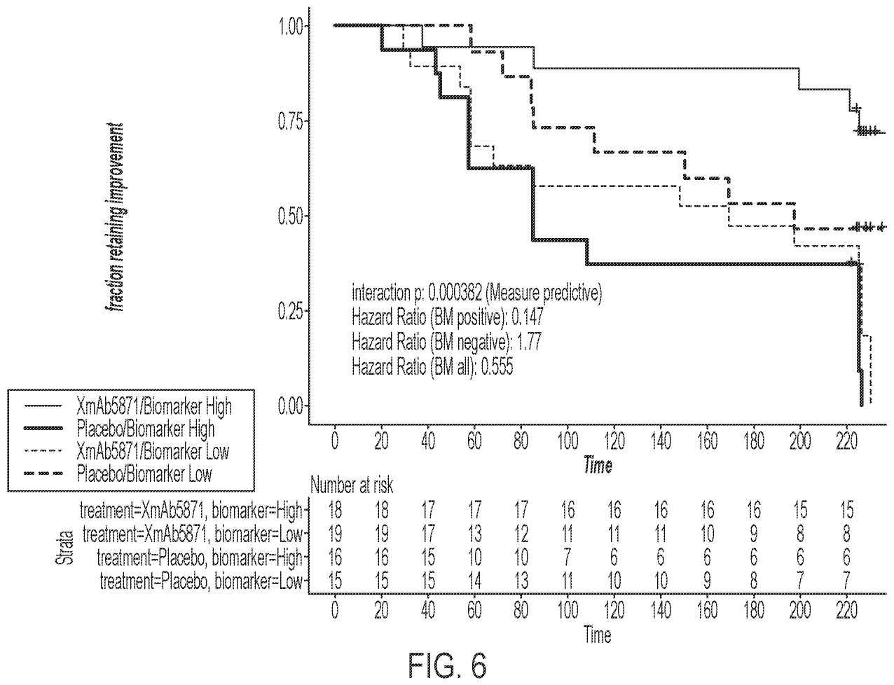

FIG. 6 shows that CD27 is a strong single gene predictor of obexelimab activity.

FIG. 6 is a cross-validated analysis. The analysis identified a cDx+ group (50% of all patients) with greatly reduced risk of flare on obexelimab using a CD27 predictive model. A five-fold cross validation framework was developed to ensure the generalizability of the results and what fraction of patients to assign as cDx+. In the placebo group, the cDx+ subjects had higher flare risk, thus cDx+ also identifies poor-prognosis patients who benefit from obexelimab.

The cDx+ group demonstrated increased obexelimab effects compared to cDx− patients over a range of clinical endpoints. Moreover, subjects who were BM− showed a worse response than BM− placebo subjects. Administration of obexelimab to BM+ patents showed a substantially increased time to LOI. The benefit of obexelimab over placebo in the cDx+ group was significantly greater than the benefit in the cDx− group for CD27. The outcomes of the placebo group in cDx− patients were better than the cDx+ group for CD27.

CD27 is therefore a strong single gene predictor of obexelimab efficacy, and/or not administering obexelimab.

### Example 3

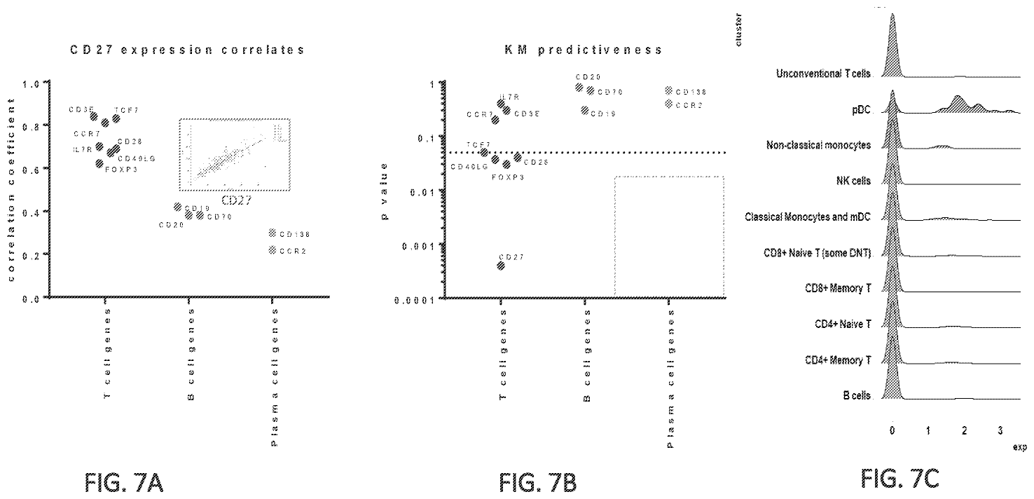

FIGS. 7A-C show the association of CD27 levels with other T cell genes and associated of APP levels with pDC.

FIG. 7A shows that CD27 is highly correlated with several T cell genes. FIG. 7B shows that biomarkers CD27, TCF7, CD40LG, FOXP3, and CD28 are predictive of antibody efficacy, as the time to LOI correlates with statistical significance (P<0.05) in a Kaplan-Meyer predictiveness analysis. Each of TCF7, CD40LG, FOXP3, and CD28 correlates with the increased expression of CD27, itself predictive of reduction in LOI for SLE subjects after obexelimab administration.

FIG. 7C shows that APP has its highest expression in pDCs in PBMC, according to the scRNAseq dataset. (Y. Kotliarov, et al. Broad immune activation underlies shared set point signatures for vaccine responsiveness in healthy individuals and disease activity in patients with lupus. Nat Med. 2020; 26(4):618-629.)

### Example 4

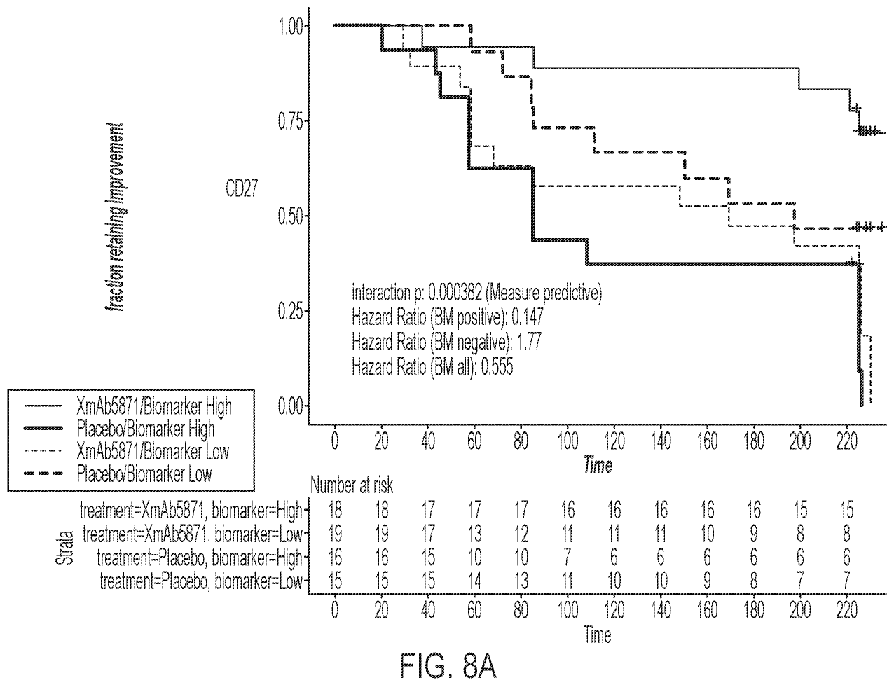

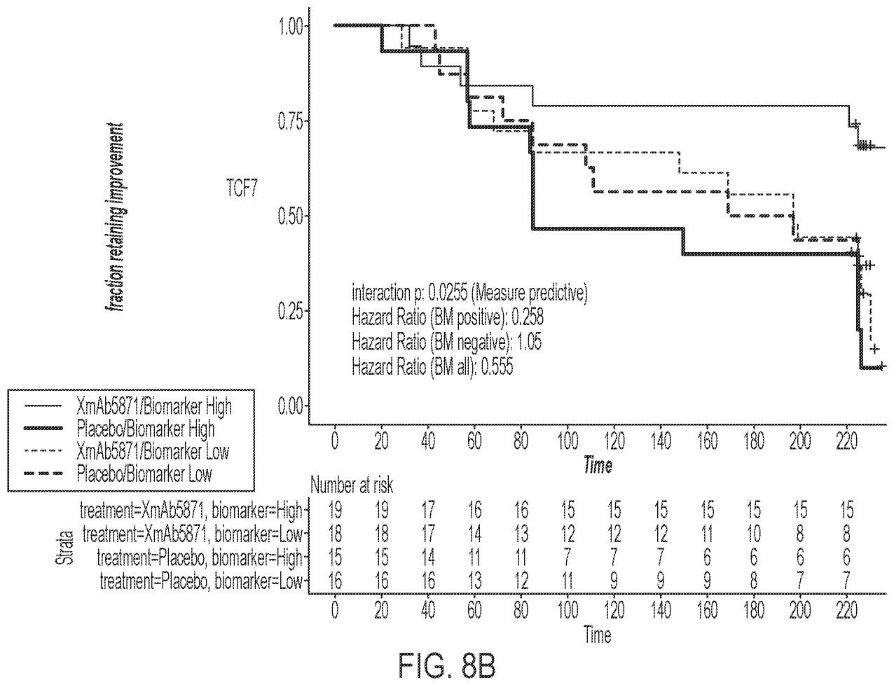

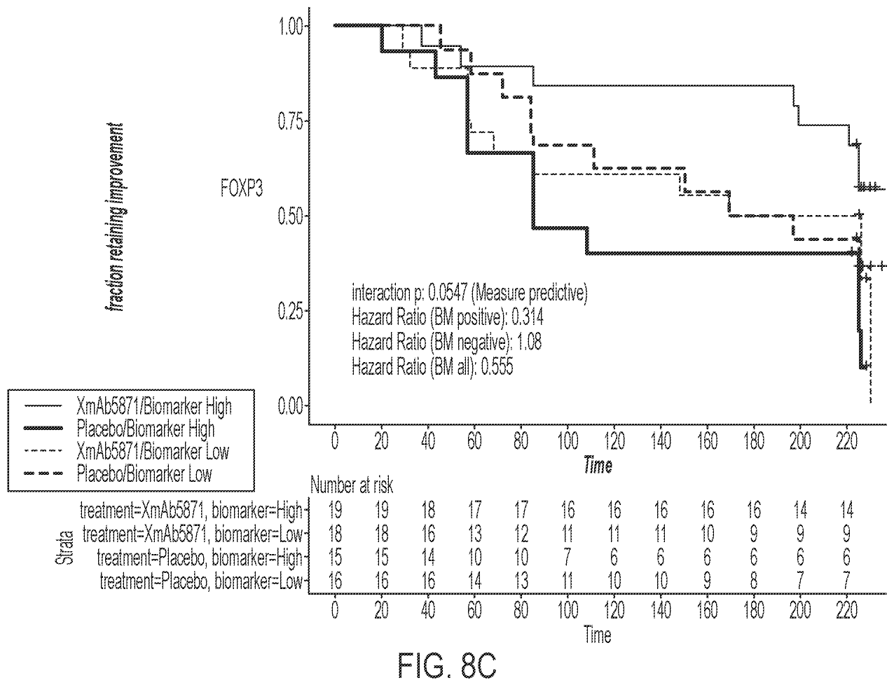

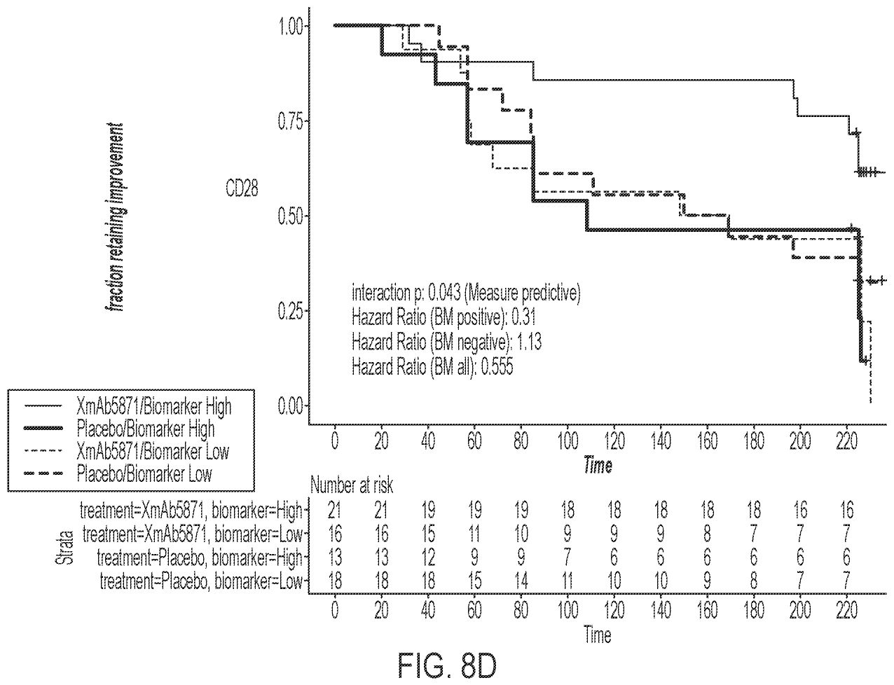

FIG. 8 depicts multiple additional genes as biomarkers of improved obexelimab treatment.

FIG. 8 shows a cross-validated analysis of CD27, TCF7, FOXP3, and CD28 expression as a predictor of treatment effectiveness for antibody and placebo subjects. The analysis identified a cDx+ group (50% of all patients) with greatly reduced risk of flare on obexelimab using a single biomarker selected from CD27, TCF7, FOXP3, or CD28 as a predictive model. A five-fold cross-validation framework was developed to ensure the generalizability of the results and what fraction of patients to assign as cDx+. In the placebo group, the CDx+ subjects had higher flare risk, thus cDX+ also identifies poor-prognosis patients who benefit from obexelimab.

The benefit of obexelimab over placebo in the cDx+ group was significantly greater than the benefit in the cDx− group for each of CD27, TCF7, FOXP3, and CD28. Further, the outcomes of the placebo group in cDx− patients were better than the cDx+ group in particular for CD27, and also for each of TCF7, FOXP3, and CD28. The absence of the biomarker can thus be used to not provide obexelimab.

### Example 5

In a blinded, placebo-controlled study, 48 healthy male volunteers were provided a single ascending dose of obexelimab. Subjects were randomized 3: obexelimab:placebo. The subjects administered obexelimab were divided into seven cohorts. Cohorts 1-7 were provided 0.03, 0.1, 0.2, 0.6, 0.03 to 10 mg/kg of obexelimab by IV infusion, respectively.

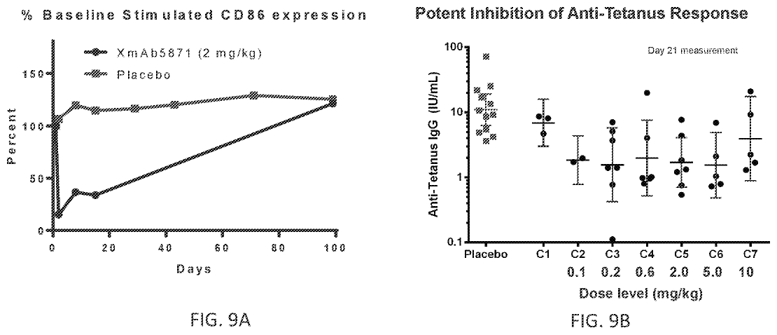

FIG. 9A depicts a substantial initial reduction in percent baseline CD86 expression compared to placebo as a function of time after obexelimab administration. Over a 100-day time course, CD86 expression returned to the placebo levels by Day 100. Reversible Suppression of CD86 upregulation and potent suppression of antibody responses was observed.

FIG. 9B anti-tetanus IgG (u/ml) shows the inhibition of anti-tetanus response for each of the separate cohorts at Day 21. Each cohort in which a dose over 0.1 mg/kg shows a statistically significant reduction in anti-tetanus IgG, with the most dose-dependent significance between 0.1 mg/kg and 5.0 mg/kg.

### Example 6

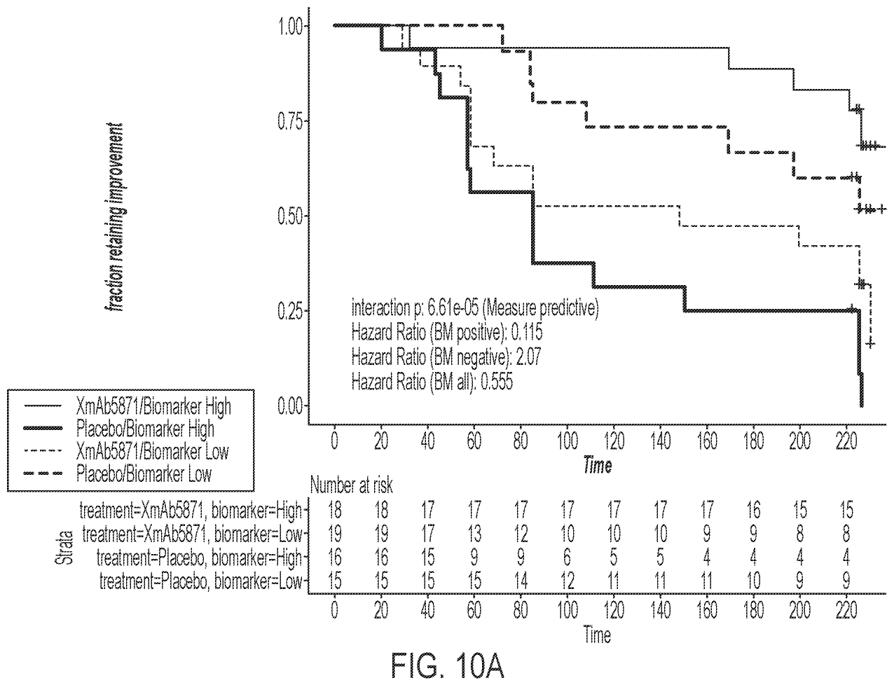

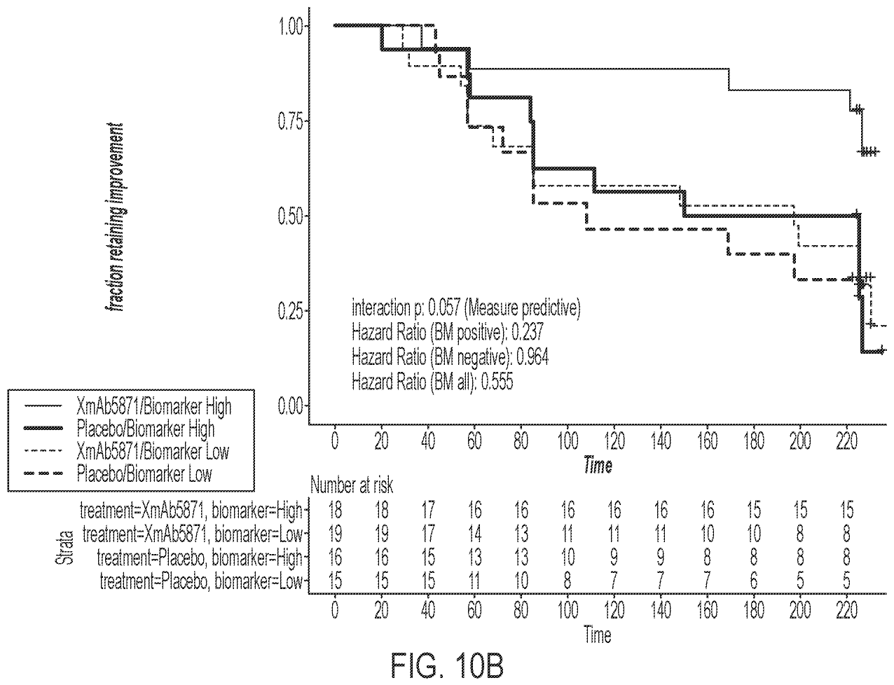

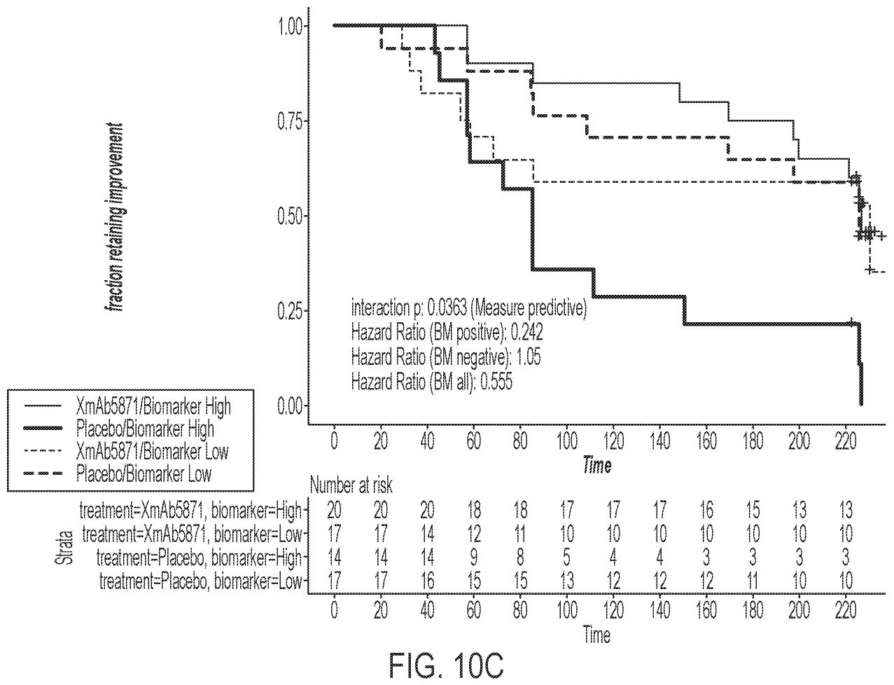

FIGS. 10A-10C show that SLE subjects with increased expression of biomarkers APP, IL-3RA (CD123), and MAP1A separately each have a statistically significant increased time to LOI if administration with obexelimab.

FIG. 10 shows a cross-validated analysis of the baseline expression level of APP, IL-3RA (CD123), and MAP1A, respectively. The analysis identified a cDx+ group (50% of all patients) with greatly reduced risk of flare on obexelimab using a single APP, IL-3RA (CD123), and MAP1A biomarker in a predictive model. A five-fold cross validation framework was developed to ensure the generalizability of the results and what fraction of patients to assign as cDx+ . In the placebo group, the CDx+ subjects had higher flare risk, thus cDX+ also identifies poor-prognosis patients who benefit from obexelimab.

The cDx+ group demonstrated increased obexelimab efficacy compared to cDx− patients for each of APP, IL-3RA (CD123), or MAP1A over a range of clinical endpoints.

The benefit of obexelimab over placebo in the cDx+ group was significantly greater than the benefit in the cDx− group for each of the single biomarkers APP, IL3RA, and MAP1A. Additionally, the outcomes of the placebo group in cDx− patients showed improvement over the cDx+ particularly for APP, and also for each of IL3RA and MAP1A. The absence of the biomarker can thus be used in a diagnosis not to provide obexelimab.

### Example 7

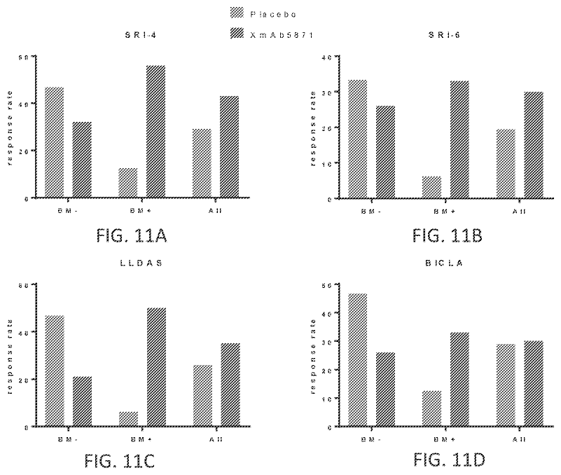

FIGS. 11A-11D show response rates are significantly enriched among cDX+ (50%) patients' four landmark endpoints at 32 weeks for patients positive with the two biomarker CD27 and APP predictive model. Moreover, the BM− subjects had a worse outcome than placebo. The landmark endpoints were for (A) SRI-4, (B) SRI-6, (C) LLDAS, and (D) BICLA.

### Example 8

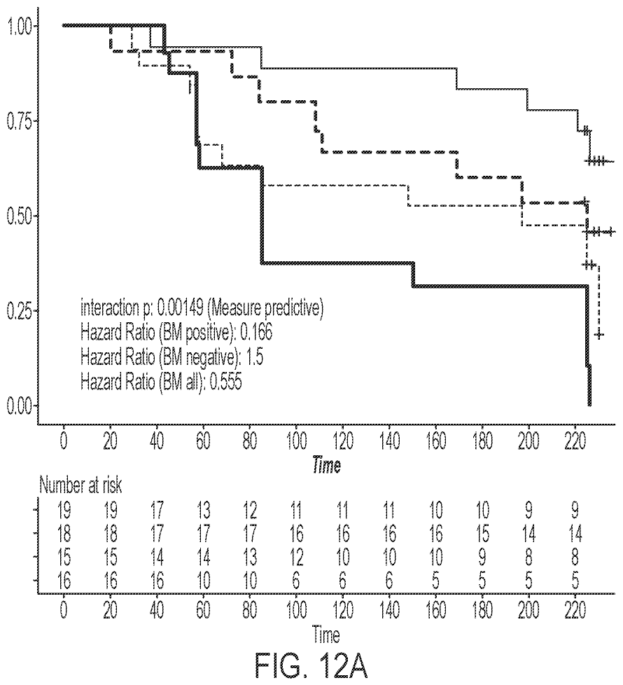

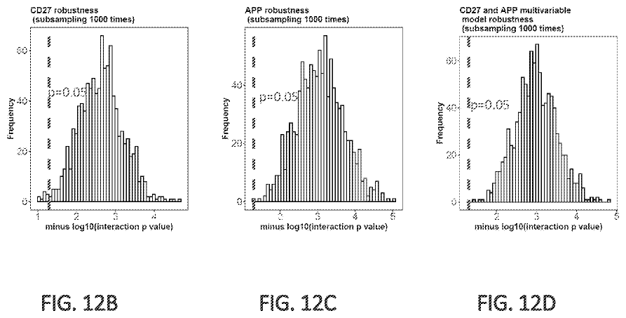

The combined CD27 and APP RNA levels were strongly predictive of efficacy. Cross-validated analyses determined that the baseline expression levels of 2 predictive biomarker genes, CD27 and APP, identified a cDx+ group (50% of all patients) with greatly reduced risk of flare on obexelimab (cross-validated HR=0.262, full data set HR=0.166) (FIG. 12). A five-fold cross validation framework was developed to ensure the generalizability of the results and to determine the number of genes needed and what fraction of patients to assign as cDx+. Classifications are based on a simple threshold for the sum of gene expression levels that have been normalized by the training sample mean and standard deviation. Conversely, in the placebo group, the CDx+ subjects had higher flare risk, thus cDX+ also identifies poor-prognosis patients who benefit from obexelimab. The cDx+ group demonstrated increased obexelimab effects compared to cDx− patients over a range of clinical endpoints (SRI-4: 56% cDx+ vs 14% cDx−, SRI-6: 33% vs 6.2%, LLDAS: 50% vs 6.2%, BICLA: 33% vs 12.5%).

FIG. 12A shows the normalized expression levels of a CD27 and APP two biomarker classification. Subjects are classified as cDX+ or cDx-, with an estimated 50% of patients assigned to each group. The benefit from obexelimab (upper solid curve) over placebo (lower solid curve) in the cDx+ group was significantly greater than the benefit in the cDx− group (blue and black dashed curves) (p=0.00149, HR cDx+=0.166 vs HR cDx−=1.5). FIG. 12A further shows a negative result clinical result for subjects that are cDx− for the combined CD27 and APP biomarkers.

The benefit of obexelimab over placebo in the cDx+ group was significantly greater than the benefit in the cDx− group in the CD27 and APP combined biomarker. In the placebo group, the cDx+ subjects had higher flare risk. cDX+ can identify poor-prognosis patients who benefit obexelimab. Additionally, the outcomes of the placebo group in cDx− patients were better than the cDx+ group. In some variations, the cDx− CD27+APP biomarker can be used as a predictor not to provide obexelimab.

FIGS. 12B-12D show the robustness of (B) CD27 single biomarker (C) APP single biomarker, and (D) CD27 + APP two-biomarker predictive model as shown by the distribution of the interaction p-value based on subsampling of 80% of the datasets, 1000 times.

CD27, the top predictive biomarker, is highly expressed by T-naïve and memory cells. Consistent with the notion that CD27 expression is important within the T-cell lineage, increased expression of other T-cell genes was also associated with reduced flare risk, including CD28 (p=0.04), TCF7(p=0.03), and FOXP3 (p=0.05). Taken together with the reduction of B cell signatures seen on treatment, this new identification of CD27 as a potential T-cell associated predictive biomarker suggests an important therapeutic role of obexelimab in suppression of B-cell and T-cell interactions.

The two biomarkers developed from whole blood transcriptomic data by RNA-sequencing identifies a biomarker positive group of patients with a high baseline resting and stem-like T-cell (CD27+, CD28+, and TCF7+) signature. These cDx+ patients had superior response rates compared to placebo across multiple clinical endpoints, suggesting that adaptive immune responses such as B-cell antigen presentation and/or T-cell costimulation may be components of obexelimab effects.

A five-fold cross validation framework was developed to ensure the generalizability of the results of the two-gene biomarker (CD27 and APP from whole blood). Clustering analyses suggested an approximately even split for biomarker positive and negative classes: the final classifier used a 50% median split of the sum of normalized CD27 and APP expression to assign patients as cDx+ or cDx−.

In one non-limiting example, a combination score can be used to determine whether to administer an antibody. The following equation depicts a two biomarker scoring model. If the measured combination was greater than or equal to 0.14, then antibody should be administered.

$$\begin{matrix}stdev & mean & \begin{matrix}Median \\ of combined\end{matrix} \\ \frac{( {X}^{CD27} - 35.88 )}{15.11} + & \frac{( {X}^{APP} - 25.36 )}{8.07} & \geq 0.14\end{matrix}$$

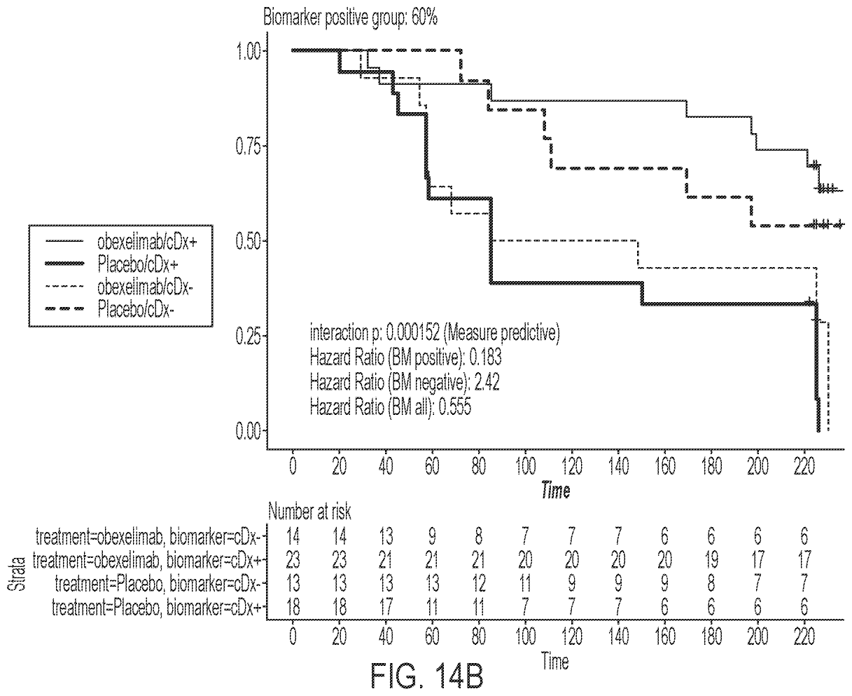

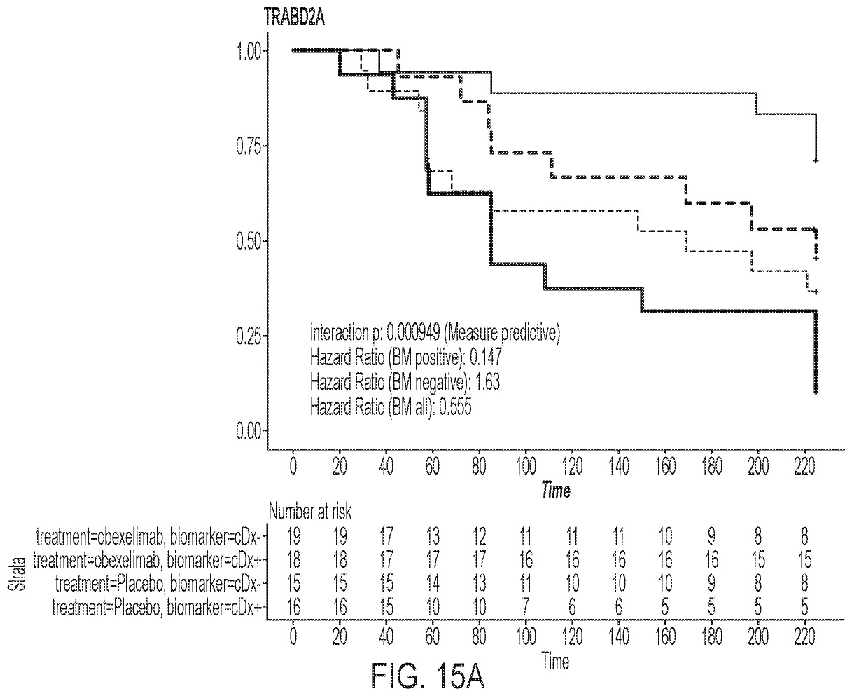

FIG. 14A and 14B show the predictability of the 40% biomarker positive group (FIG. 14A) and the 60% biomarker positive group (FIG. 14B). Specifically, FIG. 14A and 14B establish that 40% biomarker positive group and 60% biomarker positive group cutoffs both show predictability.

The range of RNA sequence expression are summarized in the Tables 2 and 3 below. Specifically, Table 2 and 3 summarize the CD27 and APP gene expression for the biomarker cohort at time points RNAseq samples were available including Screening, day 1 (D1), day 127 (D127), day 211 (D211), and day 225 (D225). No apparent trends in either CD27 or APP over time were observed.

TABLE 2

| | | Screening | D1 | D127 | D211 | D225 |
|---|---|---|---|---|---|---|
| Cohort | | (N = 68) | (N = 64) | (N = 23) | (N = 23) | (N = 24) |
| Placebo | CD27-unnorm (observed) | | | | | |
| | N | 31 | 29 | 8 | 8 | 7 |
| | N-Miss | 0 | 0 | 0 | 0 | 0 |
| | Mean | 35.788 | 38.559 | 36.012 | 30.508 | 33.966 |
| | SD | 15.547 | 17.746 | 15.569 | 7.453 | 16.977 |
| | Min | 6.086 | 9.238 | 8.835 | 19.136 | 18.241 |
| | Median (Q1, Q3) | 37.983 | 36.854 | 34.796 | 31.744 | 29.269 |
| | | (24.445, | (26.837, | (31.613, | (26.239, | (24.377, |
| | | 45.561) | 51.734) | 39.288) | 34.611) | 36.016) |
| | Max | 67.691 | 88.058 | 61.343 | 42.206 | 69.462 |
| XmAb5871 | CD27-unnorm (observed) | | | | | |
| | N | 37 | 35 | 15 | 15 | 17 |
| | N-Miss | 0 | 0 | 0 | 0 | 0 |
| | Mean | 35.964 | 30.779 | 37.983 | 37.502 | 37.200 |
| | SD | 14.943 | 12.402 | 15.779 | 17.869 | 13.985 |
| | Min | 4.360 | 10.985 | 9.429 | 14.912 | 11.456 |
| | | 37.637 | 30.437 | 38.500 | 33.351 | 39.741 |
| | Median (Q1, Q3) | (28.501, | (20.746, | (29.656, | (26.169, | (30.133, |
| | | 43.709) | 37.102) | 44.872) | 42.481) | 44.155) |
| | Max | 75.426 | 60.172 | 73.606 | 83.750 | 58.644 |

TABLE 3

| | | Screening | D1 | D127 | D211 | D225 |
|---|---|---|---|---|---|---|
| Cohort | | (N = 68) | (N = 64) | (N = 23) | (N = 23) | (N = 24) |
| Placebo | APP-unnorm (observed) | | | | | |
| | N | 31 | 29 | 8 | 8 | 7 |
| | N-Miss | 0 | 0 | 0 | 0 | 0 |
| | Mean | 25.726 | 24.240 | 26.886 | 26.620 | 26.668 |
| | SD | 10.053 | 7.888 | 12.456 | 8.592 | 5.861 |
| | Min | 2.772 | 6.600 | 6.824 | 16.910 | 15.925 |
| | | 25.265 | 23.058 | 24.531 | 25.370 | 29.170 |
| | Median (Q1, Q3) | (19.809, | (19.122, | (21.093, | (19.928, | (23.913, |
| | | 30.025) | 29.747) | 33.413) | 30.321) | 30.724) |
| | Max | 46.933 | 39.735 | 45.112 | 40.269 | 32.303 |
| XmAb5871 | APP-unnorm (observed) | | | | | |
| | N | 37 | 35 | 15 | 15 | 17 |
| | N-Miss | 0 | 0 | 0 | 0 | 0 |
| | Mean | 25.048 | 24.344 | 26.749 | 29.561 | 25.113 |
| | SD | 6.070 | 6.813 | 6.779 | 7.263 | 7.701 |
| | Min | 12.086 | 12.241 | 14.370 | 17.807 | 13.108 |
| | | 24.263 | 23.872 | 25.821 | 32.548 | 22.427 |
| | Median (Q1, Q3) | (22.185, | (19.434, | (21.295, | (23.785, | (20.264, |
| | | 27.960) | 29.307) | 31.150) | 33.667) | 29.516) |
| | Max | 40.428 | 38.127 | 39.809 | 40.809 | 39.158 |

### Example 9

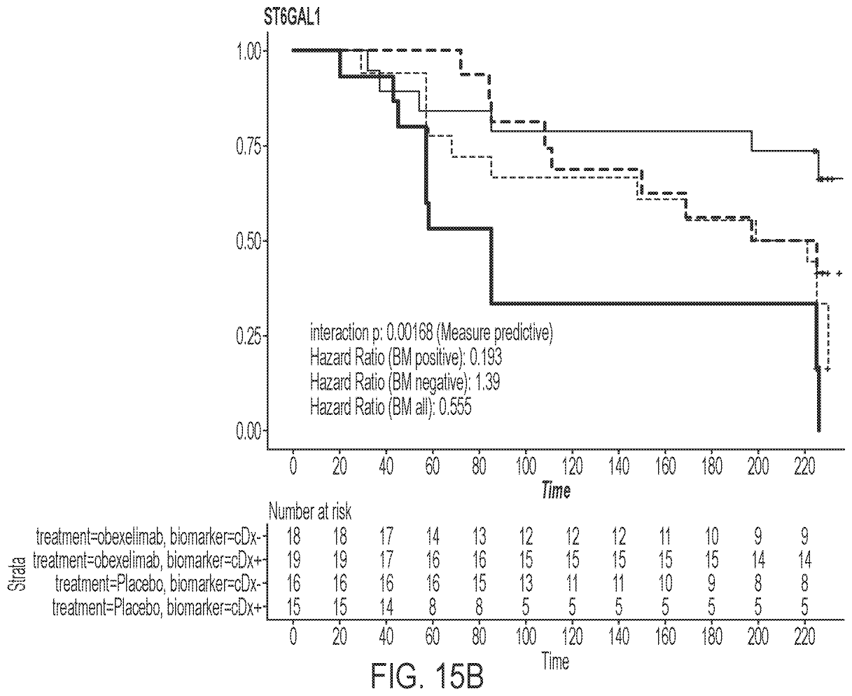

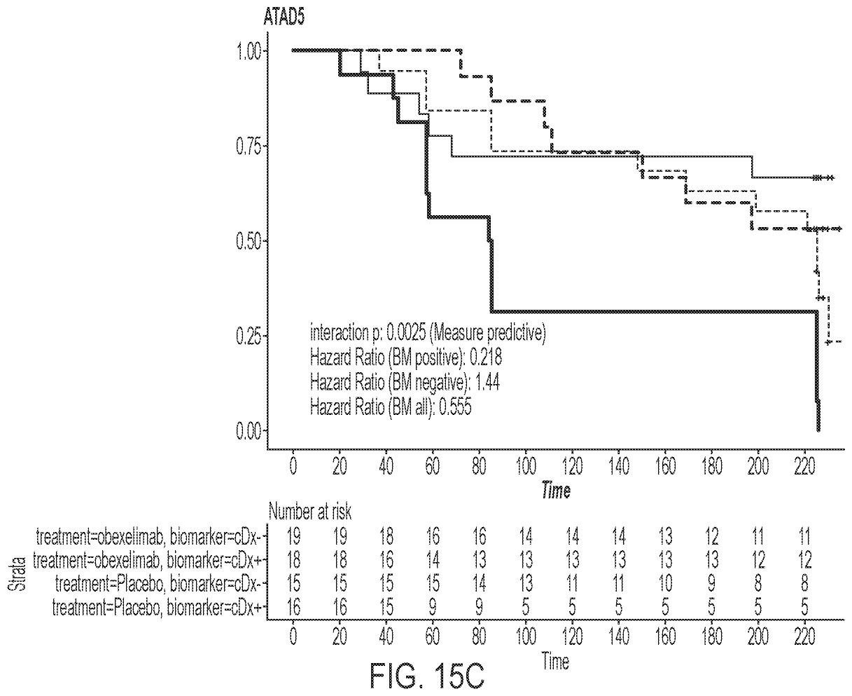

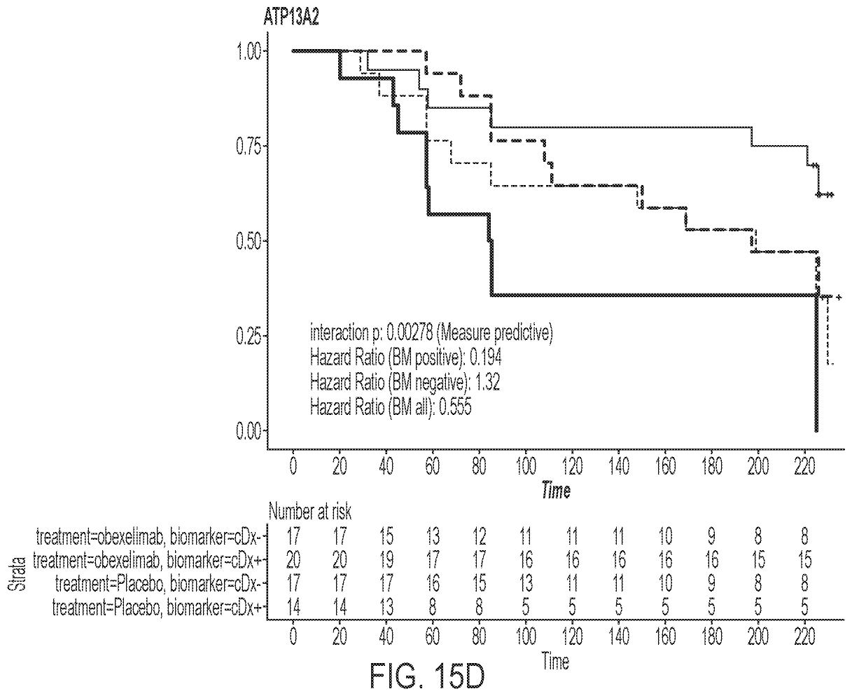

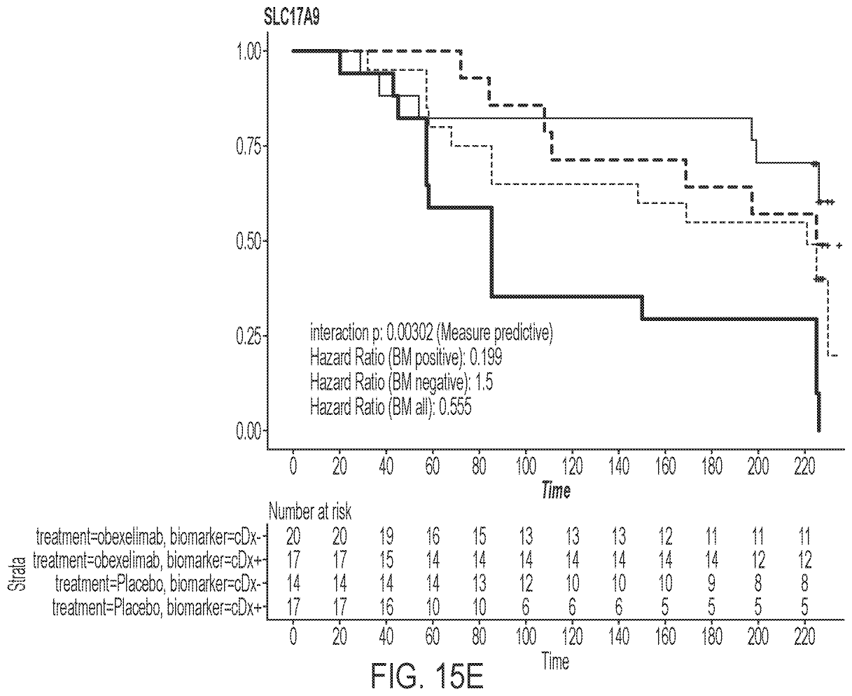

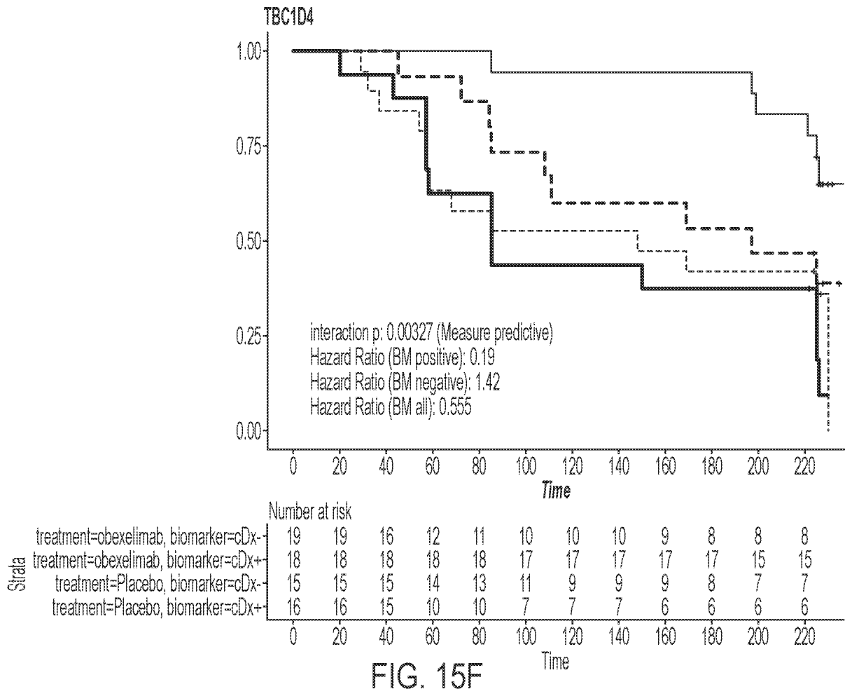

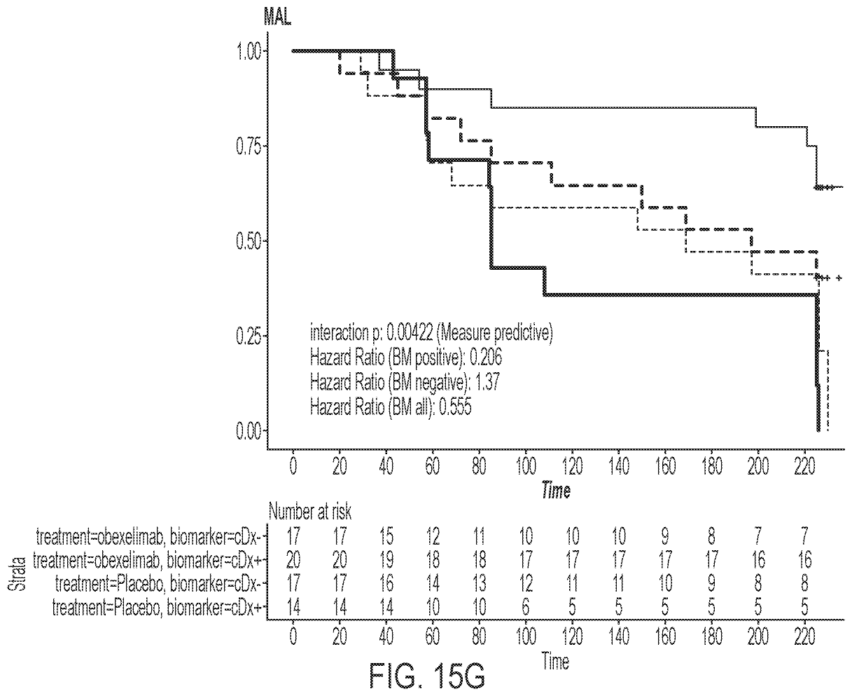

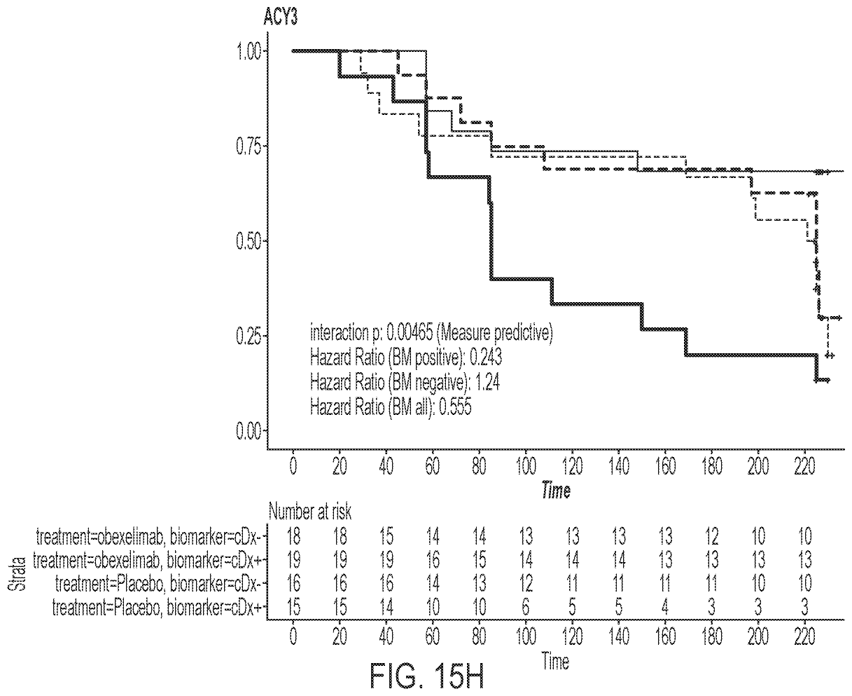

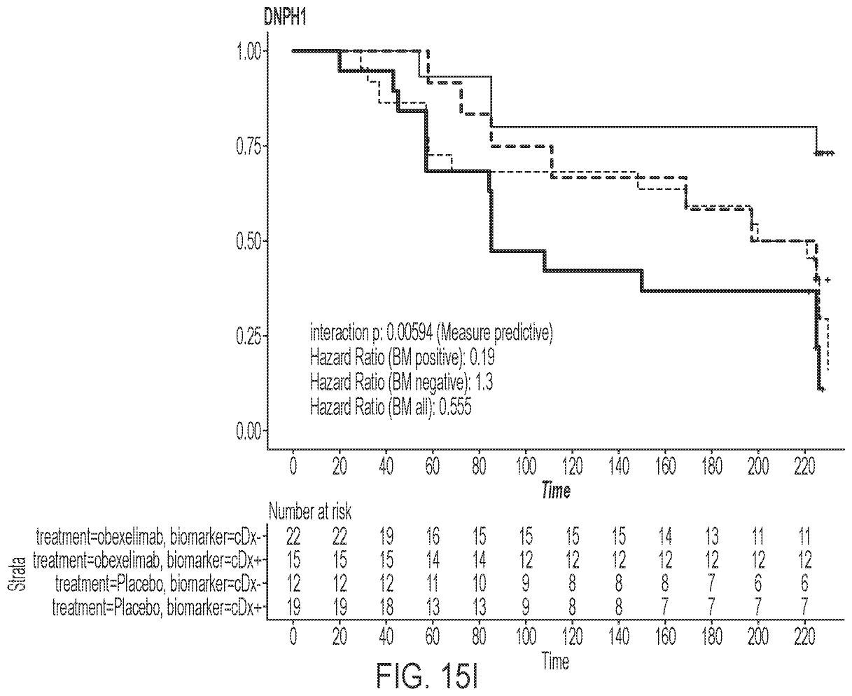

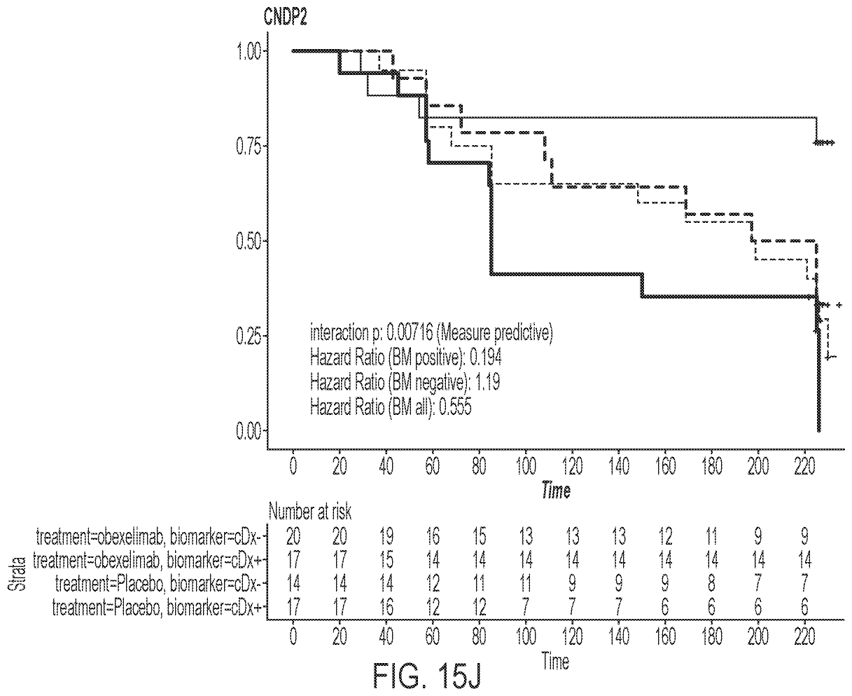

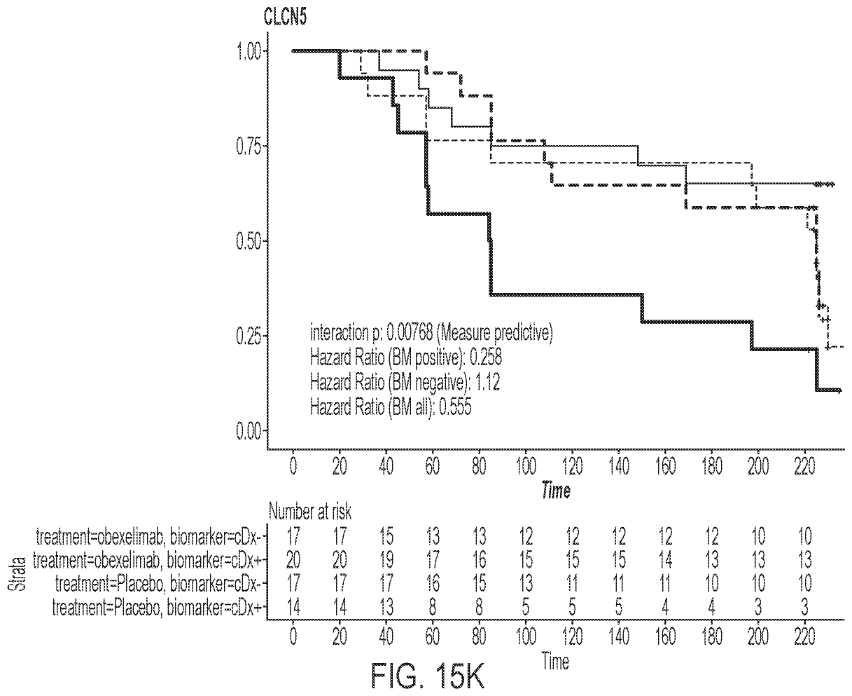

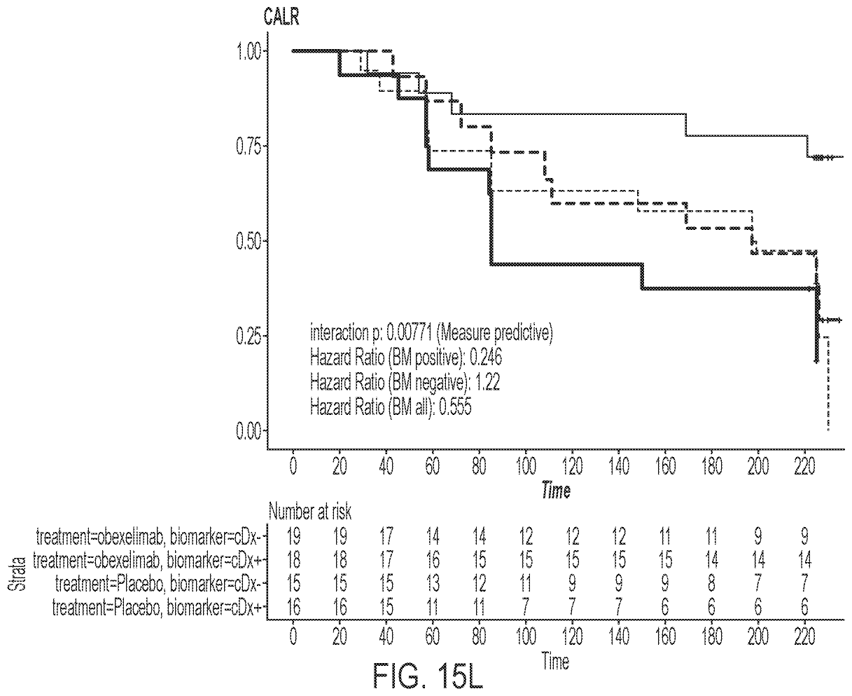

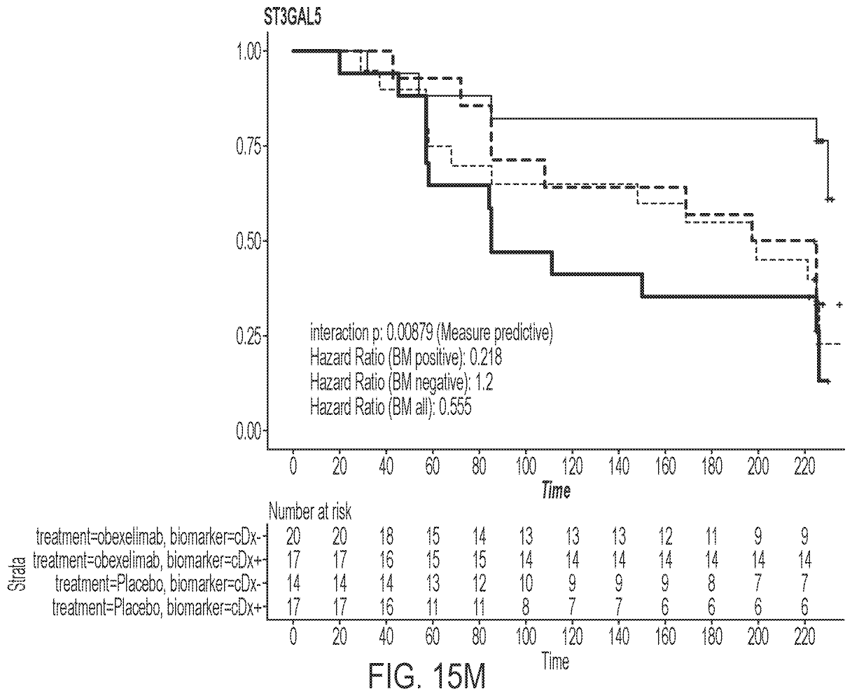

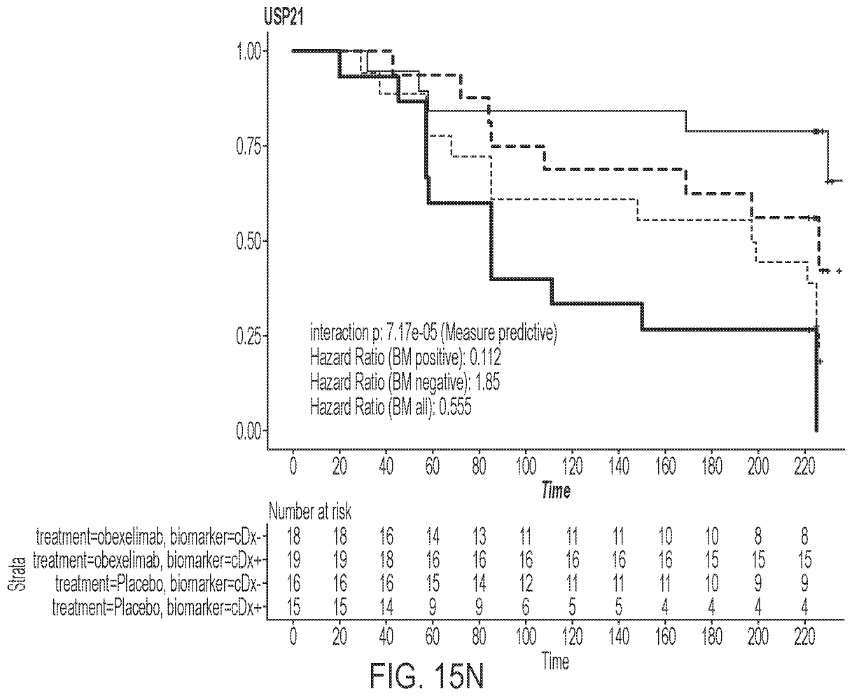

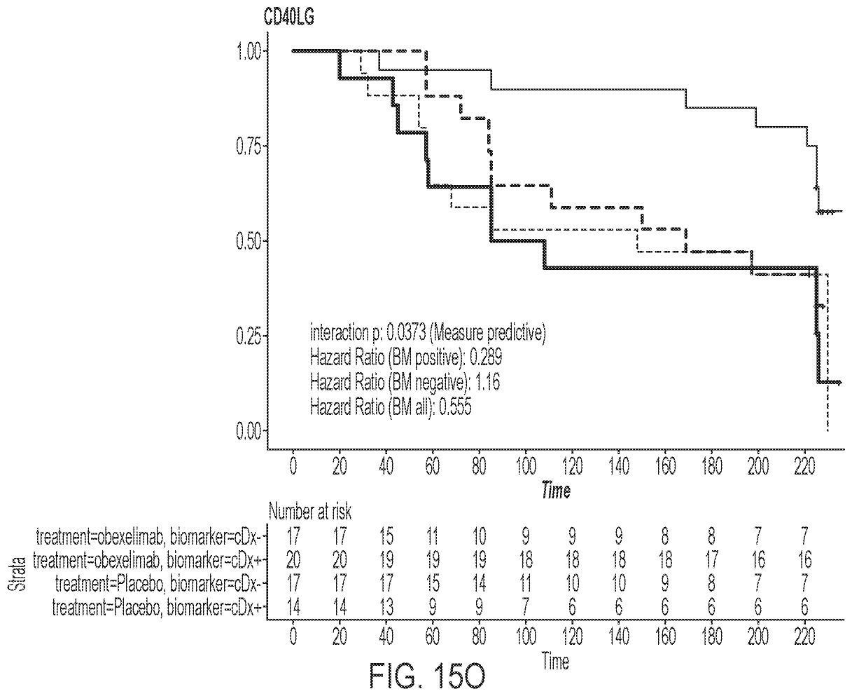

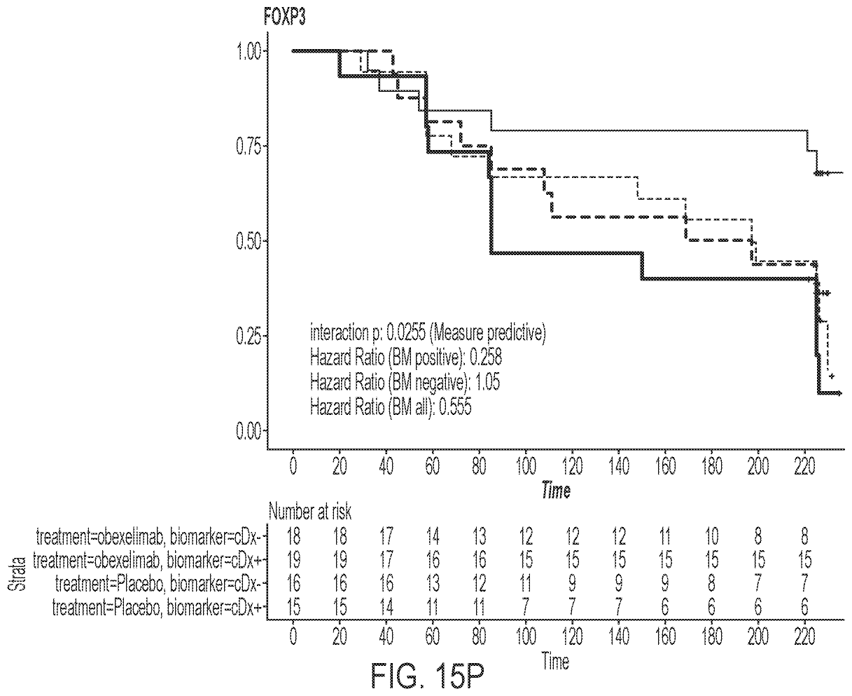

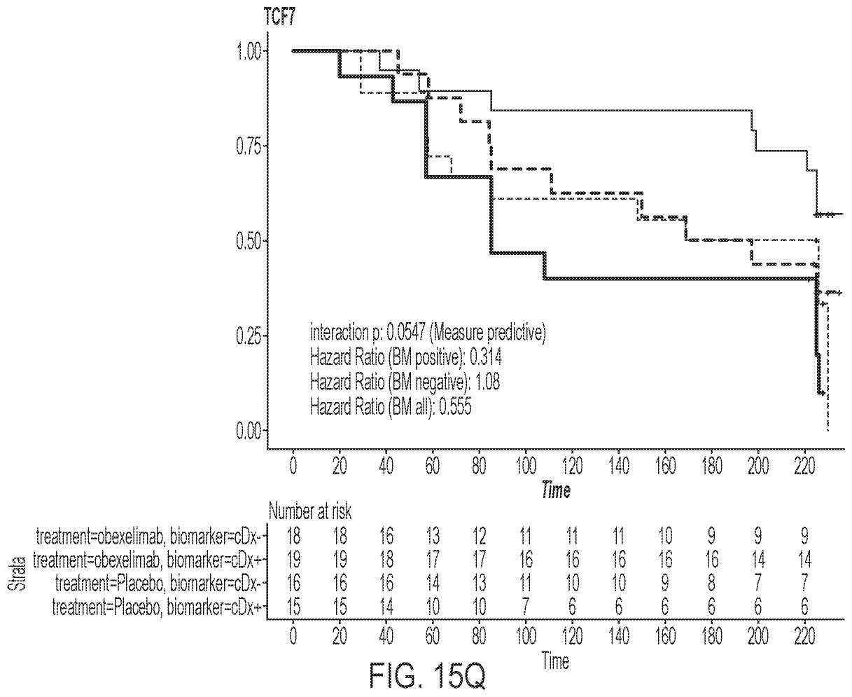

FIGS. 15A-Q depict multiple additional genes as biomarkers of improved obexelimab treatment. Specifically, FIGS. 15A-Q show a cross-validated analyses of A) TRABD2A, B) ST6GAL1, C) ATAD5, D) ATP13A2, E) SLC17A9, F) TBC1D4, G) MAL, H) ACY3, I) DNPH1, J) CNDP2, K) CLCN5, L) CALR, M) ST3GAL5, N) USP21, O) CD40LG, P) FOXP3, and Q) TCF7 expression as a predictor of treatment effectiveness for antibody and placebo subjects. The analysis identified a cDx+ group (50% of all patients) with greatly reduced risk of flare on obexelimab using a single biomarker A) TRABD2A, B) ST6GAL1, C) ATAD5, D) ATP13A2, E) SLC17A9, F) TBC1D4, G) MAL, H) ACY3, I) DNPH1, J) CNDP2, K) CLCN5, L) CALR, M) ST3GAL5, N) USP21, O) CD40LG, P) FOXP3, and Q) TCF7 as a predictive model.

The benefit of obexelimab over placebo in the cDx+ group was significantly greater than the benefit in the cDx− group for each of A) TRABD2A, B) ST6GAL1, C) ATAD5, D) ATP13A2, E) SLC17A9, F) TBC1D4, G) MAL, H) ACY3, I) DNPH1, J) CNDP2, K) CLCN5, L) CALR, M) ST3GAL5, N) USP21, O) CD40LG, P) FOXP3, and Q) TCF7 . Further, the outcomes of the placebo group in cDx− patients were better than the cDx+ group. The absence of the biomarker can thus be used to not provide obexelimab.

### Example 10

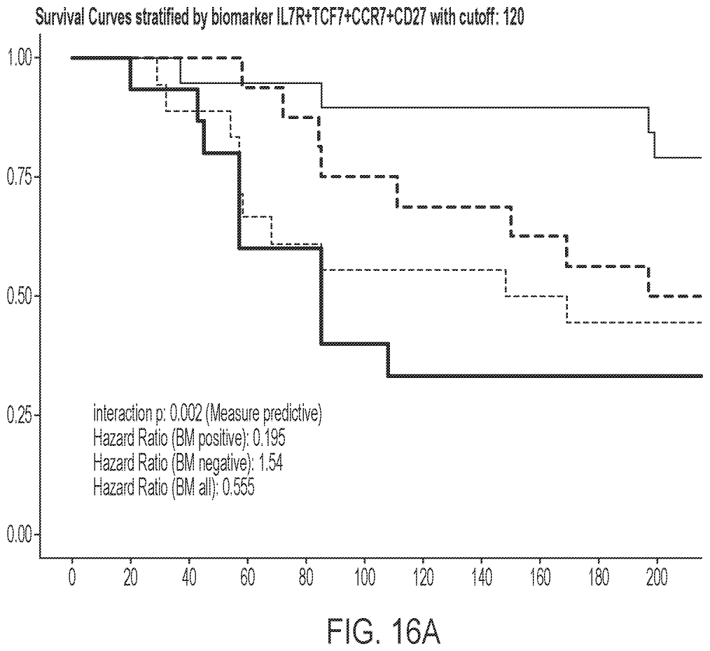

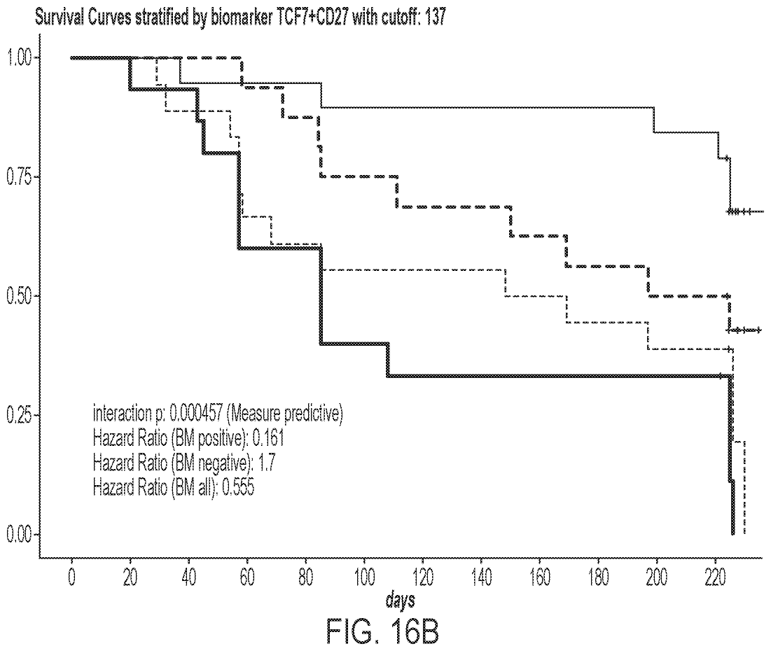

FIG. 16A-C show additional two gene, four gene, and 5 gene biomarker combinations that are suitable predictors for obexelimab activity. Specifically, FIG. 16A-C show Tim/naïve/stem cell gene signatures that may be used in combination with CD27 and TCF-7 as predictors for obexelimab activity. These gene combinations are follows: the 4-gene signature of CD27, TCF7, CCR7, and IL7R (CD127) (FIG. 16A); the 2-gene signature of CD27 and TCF7 (FIG. 16B), and the 5-gene signature of CD27, TCF7, CCR7, IL7R, and CD28 (FIG. 16C).

All cited references are herein expressly incorporated by reference in their entirety. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

### NUMBERED EMBODIMENTS

Provided here are numbered embodiments of the disclosed technology. These embodiments are illustrative only and do not limit the scope of the present disclosure or of the claims attached.

Embodiment 1. A method of treating systemic lupus erythematosus (SLE) or reducing symptoms thereof in a human subject in need thereof, comprising: determining an increased expression level of one or more biomarkers selected from CD27, TCF7, CD40LG, FOXP3, CD28, APP, IL-3RA (CD123), and MAP1A in a blood sample of the human subject; if the expression of the one or more biomarkers is increased, administering a human anti-IgG1 antibody comprising an Fc modification selected from S267E, L328F, and a combination thereof as compared to a parent IgG Fc region, wherein the numbering is according to the EU index as in Kabat.

Embodiment 2. A method of treating systemic lupus erythematosus (SLE) in a human subject in need thereof, comprising: selecting the human subject with SLE in need of such treatment by determining the increased expression of one or more biomarkers selected from CD27, TCF7, CD40LG, FOXP3, CD28, APP, IL-3RA (CD123), and MAP1A; administering a human anti-IgG1 antibody comprising an Fc modification selected from S267E, L328F, and a combination thereof as compared to a parent IgG Fc region, wherein the numbering is according to the EU index as in Kabat.

Embodiment 3. A method for treating systemic lupus erythematosus (SLE) in a human subject in thereof, wherein said method comprises: identifying said subject as having blood tissue expressing an elevated level of one or more biomarkers selected from CD27, TCF7, CD40LG, FOXP3, CD28, APP, IL-3RA (CD123), and MAP1A; and administering a human anti-IgG1 antibody comprising an Fc modification selected from S267E, L328F, and a combination thereof as compared to a parent IgG Fc region, wherein the numbering is according to the EU index as in Kabat.

Embodiment 4. The method of any one of embodiments 1-3, wherein the one or more biomarkers are selected from CD27, APP, and a combination thereof.

Embodiment 5. The method of any one of embodiments 1-4, wherein the biomarker is CD27.

Embodiment 6. The method of any one of embodiments 1-4, wherein the biomarker is APP.

Embodiment 7. The method of any one of embodiments 1-4, wherein the biomarker is the combination of CD27 and APP.

Embodiment 8. The method of any one preceding embodiments, wherein the determining or identifying step comprises administering a genotyping test to the blood sample of the subject.

Embodiment 9. The method of any one preceding embodiments, wherein the determining or identifying step comprises administering a proteomic test to the blood sample of the subject.

Embodiment 10. A method according to any one of the preceding embodiments, wherein if the expression of one or more biomarkers is not increased then the antibody is withheld from the subject.

Embodiment 11. The method of any one preceding embodiments, wherein the blood sample is whole blood.

Embodiment 12. The method of any one of the preceding embodiments, wherein the blood sample is selected from T cells, plasmablasts, and a combination thereof.

Embodiment 13. The method of any one of preceding embodiments, wherein the blood sample comprises plasmacytoid dendritic cells.

Embodiment 14. The method of any one of the preceding embodiments, wherein the antibody comprises a light chain comprising a variable region having a CDR1 comprising SEQ ID NO: 10, a CDR2 comprising SEQ ID NO: 11, and a CDR3 comprising SEQ ID NO: 12; a heavy chain comprising a variable region having a CDR1 comprising SEQ ID NO: 13, a CDR2 comprising SEQ ID NO: 14, and a CDR3 comprising SEQ ID NO: 15, and as compared to SEQ ID NO: 4; and the Fc modification is as compared to SEQ ID NO: 4, wherein the numbering is according to the EU index as in Kabat.

Embodiment 15. The method of any one of the preceding embodiments, wherein the antibody comprises: a light chain; and a heavy chain comprising an amino acid sequence of SEQ ID NO: 2 and amino acid substitutions in the Fc region S267E and L328F as compared to SEQ ID NO: 4, wherein the numbering is according to the EU index as in Kabat.

Embodiment 16. The method of any one of the preceding embodiment, wherein the antibody comprises: a light chain comprising an amino acid sequence of SEQ ID NO: 7; and a heavy chain comprising an amino acid sequence of SEQ ID NO: 9.

Embodiment 17. The method of any one of the preceding embodiments, wherein the severity of the disease within said subject is reduced and/or the days to loss of improvement (LOI) is increased.

Embodiment 18. The method of any one of the preceding embodiments, wherein the human anti-IgG1 antibody is administered subcutaneously.

Embodiment 19. The method of any one of the preceding embodiments, further comprising obtaining the blood sample from the subject.

Embodiment 20. A method of treating an autoimmune disease or reducing symptoms thereof in a human subject in need thereof, comprising: determining an increased expression level of one or more biomarkers selected from CD27, TCF7, CD40LG, FOXP3, CD28, APP, IL-3RA (CD123), and MAP1A in a blood sample of the human subject; if the expression of the one or more biomarkers is increased, administering a human anti-IgG1 antibody comprising an Fc modification selected from S267E, L328F, and a combination thereof as compared to a parent IgG Fc region, wherein the numbering is according to the EU index as in Kabat.

Embodiment 21. A method of treating an autoimmune disease in a human subject in need thereof, comprising: selecting the human subject with SLE in need of such treatment by determining the increased expression of one or more biomarkers selected from CD27, TCF7, CD40LG, FOXP3, CD28, APP, IL-3RA (CD123), and MAP1A; administering a human anti-IgG1 antibody comprising an Fc modification selected from S267E, L328F, and a combination thereof as compared to a parent IgG Fc region, wherein the numbering is according to the EU index as in Kabat.

Embodiment 22. A method for treating an autoimmune disease in a human subject in thereof, wherein said method comprises: identifying said subject as having blood tissue expressing an elevated level of one or more biomarkers selected from CD27, TCF7, CD40LG, FOXP3, CD28, APP, IL-3RA (CD123), and MAP1A; and administering a human anti-IgG1 antibody comprising an Fc modification selected from S267E, L328F, and a combination thereof as compared to a parent IgG Fc region, wherein the numbering is according to the EU index as in Kabat.

Embodiment 23. The method of one of embodiments 20-22, wherein the autoimmune disease is selected from SLE and rheumatoid arthritis.

Embodiment 24. A method of improving therapeutic efficacy for treatment of systemic lupus erythematosus (SLE), comprising: determining the expression of one or more biomarkers selected from CD27, TCF7, CD40LG, FOXP3, CD28, APP, IL-3RA (CD123), and MAP1A in a subject having SLE; wherein an increase of the expression of the one or more biomarkers indicates the efficacy of a human anti-IgG1 antibody comprising an Fc modification selected from S267E, L328F, and a combination thereof as compared to a parent IgG Fc region, wherein the numbering is according to the EU index as in Kabat, in the subject.

Embodiment 25. A method of determining susceptibility to treatment for systemic lupus erythematosus (SLE) in a human subject in need thereof, comprising: determining the expression of one or more biomarkers selected from CD27, TCF7, CD40LG, FOXP3, CD28, APP, IL-3RA (CD123), and MAP1A in the subject; wherein an increase of the expression of the one or more biomarkers indicates the efficacy of a human anti-IgG1 antibody comprising an Fc modification selected from S267E, L328F, and a combination thereof as compared to a parent IgG Fc region, wherein the numbering is according to the EU index as in Kabat, in the subject.

Embodiment 26. The method of any one of embodiments 24 or 25, wherein an increase of the expression of one or more biomarkers selected from CD27, TCF7, CD40LG, FOXP3, CD28, APP, IL-3RA (CD123), and MAP1A in the subject indicates that the human anti-IgG1 antibody will be efficacious in the subject.

Embodiment 27. The method of any one of embodiments 20-26, wherein the one or more biomarkers are selected from CD27, APP, and a combination thereof.

Embodiment 28. The method of any one of embodiments 20-27, wherein the biomarker is CD27.

Embodiment 29. The method of any one of embodiments 20-27, wherein the biomarker is APP.

Embodiment 30. The method of any one of embodiments 20-27, wherein the biomarker is the combination of CD27 and APP.

Embodiment 31. The method of any one of embodiments 20-30, wherein the determining or identifying step comprises administering a genotyping test to the blood sample of the subject.

Embodiment 32. The method of any one of embodiments 20-30, wherein the determining or identifying step comprises administering a proteomic test to the blood sample of the subject.

Embodiment 33. A method according to any one of embodiments 20-32, wherein if the expression of one or more biomarkers is not increased then the antibody is withheld from the subject.

Embodiment 34. The method of any one of embodiments 20-33, wherein the blood sample is whole blood.

Embodiment 35. The method of any one of embodiments 20-33, wherein the blood sample is selected from T cells, plasmablasts, and a combination thereof.

Embodiment 36. The method of any one of embodiments 20-33, wherein the blood sample comprises plasmacytoid dendritic cells.

Provided below are further numbered embodiments of the disclosed technology.

Further embodiment 1. A method of treating an autoimmune disease or reducing symptoms thereof in a human subject in need thereof, comprising: determining an increased expression level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a blood sample of the human subject; and if the expression of the one or more biomarkers is increased, administering a human anti-CD19 antibody comprising an Fc modification selected from S267E, L328F, and a combination thereof as compared to a parent IgG Fc region, wherein the numbering is according to the EU index as in Kabat.

Further embodiment 2. A method of treating autoimmune disease or reducing symptoms thereof, comprising: selecting the human subject with the autoimmune disease in need of such treatment by determining the increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21; and administering a human anti-CD19 antibody comprising an Fc modification selected from S267E, L328F, and a combination thereof as compared to a parent IgG Fc region, wherein the numbering is according to the EU index as in Kabat.

Further embodiment 3. A method of treating autoimmune disease or reducing symptoms thereof, wherein said method comprises: identifying said subject as having an increased expression level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21; and administering a human anti-CD19 antibody comprising an Fc modification selected from S267E, L328F, and a combination thereof as compared to a parent IgG Fc region, wherein the numbering is according to the EU index as in Kabat.

Further embodiment 4. A method of selecting one or more human subjects for treating an autoimmune disease or reducing symptoms thereof, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more subjects; and to the subjects having increased expression, administering a human anti-CD19 antibody comprising an Fc modification selected from S267E, L328F, and a combination thereof as compared to a parent IgG Fc region, wherein the numbering is according to the EU index as in Kabat.

Further embodiment 5. A method of treating SLE or reducing symptoms thereof in a human subject in need thereof, comprising: determining an increased expression level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a blood sample of the human subject; if the expression of the one or more biomarkers is increased, administering a human anti-CD19 antibody comprising an Fc modification selected from S267E, L328F, and a combination thereof as compared to a parent IgG Fc region, wherein the numbering is according to the EU index as in Kabat.

Further embodiment 6. A method of treating SLE in a human subject in need thereof, comprising: selecting the human subject with SLE in need of such treatment by determining the increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21; administering a human anti-CD19 antibody comprising an Fc modification selected from S267E, L328F, and a combination thereof as compared to a parent IgG Fc region, wherein the numbering is according to the EU index as in Kabat.

Further embodiment 7. A method for treating SLE in a human subject in thereof, wherein said method comprises: identifying said subject as having an increased expression level of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21; and administering a human anti-CD19 antibody comprising an Fc modification selected from S267E, L328F, and a combination thereof as compared to a parent IgG Fc region, wherein the numbering is according to the EU index as in Kabat.

Further embodiment 8. A method of selecting one or more human subjects for treating SLE, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more subjects; and to the subjects having increased expression, administering a human anti-CD19 antibody comprising an Fc modification selected from S267E, L328F, and a combination thereof as compared to a parent IgG Fc region, wherein the numbering is according to the EU index as in Kabat.

Alternatively, the biomarkers in further embodiments 1-8 may be: (a) one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127); (b) one or more biomarkers selected from CD27, TCF7, CD40LG, FOXP3, and CD28, and/or one or more biomarkers selected from APP, IL-3RA (CD123), and MAP1A; (c) CD27 and TCF; (d) CD27, TCF7, CCR7, and IL7R; (e) CD27, TCF7, CCR7, IL7R, and CD28; (f) CD27, TCF7, CD40LG, FOXP3, CD28; and (g) APP, IL-3RA (CD123), and MAP1A.

Further embodiment 9. The method of any one of further embodiments 1-8, wherein the one or more biomarkers are selected from CD27, TCF7, CD40LG, FOXP3, CD28, APP, IL-3RA (CD123), MAP1A.

Further embodiment 10. The method of any one of further embodiments 1-8, wherein the one or more biomarkers are selected from CD27, TCF7, CD40LG, FOXP3, CD28.

Further embodiment 11. The method of any one of further embodiments 1-8, wherein the one or more biomarkers are selected from TRABD2A, ST6GAL1, ATAD5, ATP13A2, SLC17A9, TBC1D4, MAL, ACY3, DNPH1, CNDP2, CLCN5, CALR, ST3GAL5, and USP21.

Further embodiment 12. The method of any one of further embodiments 1-8, wherein the one or more biomarkers are selected from CD27 and APP.

Further embodiment 13. The method of any one of further embodiments 1-8, wherein the biomarker is CD27.

Further embodiment 14. The method of any one of further embodiments 1-8, wherein the biomarker is APP.

Further embodiment 15. The method of any one of further embodiments 1-8, wherein the biomarker is the combination of CD27 and APP.

Further embodiment 16. The method of any one of the preceding further embodiments, wherein the determining or identifying step comprises administering a genotyping test to the blood sample of the subject.

Further embodiment 17. The method of any one of the preceding further embodiments, wherein the determining or identifying step comprises administering a proteomic test to the blood sample of the subject.

Further embodiment 18. A method according to any one of the preceding further embodiments, wherein if the expression of one or more biomarkers is not increased then the antibody is withheld from the subject.

Further embodiment 19. The method of any one of the preceding further embodiments, wherein the blood sample is whole blood.

Further embodiment 20. The method of any one of the preceding further embodiments, wherein the blood sample is selected from T cells, plasmablasts, and a combination thereof.

Further embodiment 21. The method of any one of the preceding further embodiments, wherein the blood sample comprises plasmacytoid dendritic cells.

Further embodiment 22. The method of any one of the preceding further embodiments, wherein the antibody comprises a light chain comprising a variable region having a CDR1 comprising SEQ ID NO: 10, a CDR2 comprising SEQ ID NO: 11, and a CDR3 comprising SEQ ID NO: 12; a heavy chain comprising a variable region having a CDR1 comprising SEQ ID NO: 13, a CDR2 comprising SEQ ID NO: 14, and a CDR3 comprising SEQ ID NO: 15, and as compared to SEQ ID NO: 4; and the Fc modification is as compared to SEQ ID NO: 4, wherein the numbering is according to the EU index as in Kabat.

Further embodiment 23. The method of any one of the preceding further embodiments, wherein the antibody comprises a light chain; and a heavy chain comprising an amino acid sequence of SEQ ID NO: 2 and amino acid substitutions in the Fc region S267E and L328F as compared to SEQ ID NO: 4, wherein the numbering is according to the EU index as in Kabat.

Further embodiment 24. The method of any one of the preceding further embodiments, wherein the antibody comprises a light chain comprising an amino acid sequence of SEQ ID NO: 7; and a heavy chain comprising an amino acid sequence of SEQ ID NO: 9.

Further embodiment 25. The method of any one of the preceding further embodiments, wherein the severity of the disease within said subject is reduced and/or the days to loss of improvement (LOI) is increased.

Further embodiment 26. The method of any one of the preceding further embodiments, wherein the human anti-CD19 antibody is administered subcutaneously.

Further embodiment 27. The method of any one of the preceding further embodiments, further comprising obtaining the blood sample from the subject.

Further embodiment 28. The method of one of further embodiments 1-4 or 9-27, wherein the autoimmune disease is selected from SLE and rheumatoid arthritis.

Further embodiment 29. A method of improving therapeutic efficacy for treatment of an autoimmune disease, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a subject having the autoimmune disease; wherein an increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

Further embodiment 30. A method of determining susceptibility to treatment for an autoimmune disease in a human subject in need thereof, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the subject; wherein an increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

Further embodiment 31. A method of selecting one or more human subjects with increased responsiveness to treatment of an autoimmune disease, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more subjects, wherein the increased expression of the one or more biomarkers corresponds to an increase in responsiveness in the subject.

Further embodiment 32. A method of improving therapeutic efficacy for treatment of SLE, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a subject having SLE; wherein an increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

Further embodiment 33. A method of determining susceptibility to treatment for SLE in a human subject in need thereof, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21in the subject; wherein an increase of the expression of the one or more biomarkers indicates the efficacy of a human anti-CD19 antibody comprising an Fc modification selected from S267E, L328F, and a combination thereof as compared to a parent IgG Fc region, wherein the numbering is according to the EU index as in Kabat, in the subject.

Further embodiment 34. A method of selecting one or more human subjects with increased responsiveness to SLE treatment, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more subjects, wherein the populations, wherein the increased expression of the one or more biomarkers corresponds to an increase in responsiveness in the subject.

Alternatively, the biomarkers in further embodiments 29-34 may be: (a) one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127); (b) one or more biomarkers selected from CD27, TCF7, CD40LG, FOXP3, and CD28, and/or one or more biomarkers selected from APP, IL-3RA (CD123), and MAP1A; (c) CD27 and TCF; (d) CD27, TCF7, CCR7, and IL7R; (e) CD27, TCF7, CCR7, IL7R, and CD28; (f) CD27, TCF7, CD40LG, FOXP3, CD28; and (g) APP, IL-3RA (CD123), and MAP1A.

Further embodiment 35. The method of any one of further embodiments 29-34, wherein the increase of the expression of one or more biomarkers selected from CD27, TCF7, CD40LG, FOXP3, CD28, APP, IL-3RA (CD123), and MAP1A in the subject indicates that the human anti-CD19 antibody will be efficacious in the subject.

Further embodiment 36. The method of any one of further embodiments 29-34, wherein the one or more biomarkers are selected from CD27, TCF7, CD40LG, FOXP3, CD28.

Further embodiment 37. The method of any one of further embodiments 29-34, wherein the one or more biomarkers are selected from TRABD2A, ST6GAL1, ATAD5, ATP13A2, SLC17A9, TBC1D4, MAL, ACY3, DNPH1, CNDP2, CLCN5, CALR, ST3GAL5, and USP21.

Further embodiment 38. The method of any one of further embodiments 29-34, wherein the one or more biomarkers are selected from CD27 and APP.

Further embodiment 39. The method of any one of further embodiments 29-34, wherein the biomarker is CD27.

Further embodiment 40. The method of any one of further embodiments 29-34, wherein the biomarker is APP.

Further embodiment 41. The method of any one of further embodiments 29-34, wherein the biomarker is the combination of CD27 and APP.

Further embodiment 42. The method of any one of further embodiments 29-41, wherein the determining or identifying step comprises administering a genotyping test to the blood sample of the subject.

Further embodiment 43. The method of any one of further embodiments 29-41, wherein the determining or identifying step comprises administering a proteomic test to the blood sample of the subject.

Further embodiment 44. A method according to any one of further embodiments 29-43, wherein if the expression of one or more biomarkers is not increased then the antibody is withheld from the subject.

Further embodiment 45. The method of any one of further embodiments 29-44, wherein the blood sample is whole blood.

Further embodiment 46. The method of any one of further embodiments 29-44, wherein the blood sample is selected from T cells, plasmablasts, and a combination thereof.

Further embodiment 47. The method of any one of further embodiments 29-44, wherein the blood sample comprises plasmacytoid dendritic cells.

Further embodiment 48. An in vitro method of improving therapeutic efficacy for treatment of an autoimmune disease, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from a subject having the autoimmune disease, wherein an increase of the expression of the one or more biomarkers indicates the efficacy of a human anti-CD19 antibody in the subject.

Further embodiment 49. An in vitro method of determining susceptibility to treatment for an autoimmune disease in a human subject in need thereof, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from the subject, wherein the subject has been treated with a human anti-CD19 antibody, wherein an increase of the expression of the one or more biomarkers indicates the efficacy of a human anti-CD19 antibody in the subject.

Further embodiment 50. An in vitro method of identifying one or more human subjects with increased responsiveness to treatment of an autoimmune disease, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from the one or more subjects having been treated with a human anti-CD19 antibody, wherein the increased expression of the one or more biomarkers corresponds to an increase in responsiveness in the subject.

Further embodiment 51. The in vitro method of any one of further embodiments 48-50, comprising: determining expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from the subject prior to treatment with the antibody or in a sample from a subject having the autoimmune disease; and comparing the expression of the one or more biomarkers to determine increased expression.

Further embodiment 52. An in vitro method of determining susceptibility to treatment for SLE in a human subject in need thereof, comprising: determining the expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from the SLE patient having been treated with a human anti-CD19 antibody, wherein an increase of the expression of the one or more biomarkers indicates the efficacy of a human anti-CD19 antibody in the subject.

Further embodiment 53. An in vitro method of identifying one or more human subjects with increased responsiveness to SLE treatment, comprising: determining increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in the one or more SLE subjects having been treated a human anti-CD19 antibody, wherein the populations, wherein the increased expression of the one or more biomarkers corresponds to an increase in responsiveness in the subject.

Further embodiment 54. The in vitro method of further embodiments 52 or 53, comprising: determining expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21 in a sample from the subject prior to treatment with the antibody or in a sample from a subject having SLE; and comparing the expression of the one or more biomarkers to determine increased expression.

Alternatively, the biomarkers in further embodiments 48-54 may be: (a) one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127); (b) one or more biomarkers selected from CD27, TCF7, CD40LG, FOXP3, and CD28, and/or one or more biomarkers selected from APP, IL-3RA (CD123), and MAP1A; (c) CD27 and TCF; (d) CD27, TCF7, CCR7, and IL7R; (e) CD27, TCF7, CCR7, IL7R, and CD28; (f) CD27, TCF7, CD40LG, FOXP3, CD28; and (g) APP, IL-3RA (CD123), and MAP1A.

Further embodiment 55. The in vitro method of any one of further embodiments 48-54, wherein the anti-CD19 antibody comprises an Fc modification selected from S267E, L328F, and a combination thereof as compared to a parent IgG Fc region, wherein the numbering is according to the EU index as in Kabat.

Further embodiment 56. The in vitro method of any one of further embodiments 48-55, wherein an increase of the expression of one or more biomarkers selected from CD27, TCF7, CD40LG, FOXP3, CD28, APP, IL-3RA (CD123), and MAP1A in the subject indicates that the human anti-CD19 antibody will be efficacious in the subject.

Further embodiment 57. The in vitro method of any one of further embodiments 48-55, wherein the one or more biomarkers are selected from CD27, TCF7, CD40LG, FOXP3, CD28.

Further embodiment 58. The in vitro method of any one of further embodiments 48-55, wherein the one or more biomarkers are selected from TRABD2A, ST6GAL1, ATAD5, ATP13A2, SLC17A9, TBC1D4, MAL, ACY3, DNPH1, CNDP2, CLCN5, CALR, ST3GAL5, and USP21.

Further embodiment 59. The in vitro method of any one of further embodiments 48-55, wherein the one or more biomarkers are selected from CD27 and APP.

Further embodiment 60. The in vitro method of any one of further embodiments 48-55, wherein the biomarker is CD27.

Further embodiment 61. The in vitro method of any one of further embodiments 48-55, wherein the biomarker is APP.

Further embodiment 62. The in vitro method of any one of further embodiments 48-55, wherein the biomarker is the combination of CD27 and APP.

Further embodiment 63. The in vitro method of any one of further embodiments 48-62, wherein the sample is a blood sample.

Further embodiment 64. The in vitro method of further embodiment

63, wherein the blood comprises T cells, plasmablasts, and a combination thereof.

Further embodiment 65. The in vitro method of further embodiment 63, wherein the blood sample comprises plasmacytoid dendritic cells.

Further embodiment 66. Use of a therapeutically effective amount of an anti-CD19 antibody for treating systemic lupus erythematous (SLE) in a human subject having increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21, wherein the anti-CD19 antibody comprises an Fc modification selected from S267E, L328F, and a combination thereof as compared to a parent IgG Fc region, wherein the numbering is according to the EU index as in Kabat.

Further embodiment 67. Use of a therapeutically effective amount of an anti-CD19 antibody in the manufacture of a medicament for treating systemic lupus erythematous (SLE) in a human subject having increased expression of one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, and USP21, wherein the anti-CD19 antibody comprises an Fc modification selected from S267E, L328F, and a combination thereof as compared to a parent IgG Fc region, wherein the numbering is according to the EU index as in Kabat.

Alternatively, the biomarkers in further embodiments 66 or 67 may be: (a) one or more biomarkers selected from CD27, TCF7, CD40LG, FOXP3, CD28, APP, IL-3RA (CD123), MAP1A, TRABD2A, ST6GAL1, ATAD5, ATP13A2, SLC17A9, TBC1D4, MAL, ACY3, DNPH1, CNDP2, CLCN5, CALR, ST3GAL5, USP21, and IL7R (CD127); (b) one or more biomarkers selected from CD27, TCF7, CD40LG, FOXP3, and CD28, and/or one or more biomarkers selected from APP, IL-3RA (CD123), and MAP1A; (c) CD27 and TCF; (d) CD27, TCF7, CCR7, and IL7R; (e) CD27, TCF7, CCR7, IL7R, and CD28; (f) CD27, TCF7, CD40LG, FOXP3, CD28; and (g) APP, IL-3RA (CD123), and MAP1A.

Further embodiment 68. The use of further embodiments 66 or 67, wherein the one or more biomarkers are selected from CD27, TCF7, CD40LG, FOXP3, and CD28.

Further embodiment 69. The use of further embodiments 66 or 67, wherein the one or more biomarkers are selected from TRABD2A, ST6GAL1, ATAD5, ATP13A2, SLC17A9, TBC1D4, MAL, ACY3, DNPH1, CNDP2, CLCN5, CALR, ST3GAL5, and USP21.

Further embodiment 70. The use of further embodiments 66 or 67, wherein the one or more biomarkers are selected from CD27 and APP.

Further embodiment 71. The use of further embodiments 66 or 67, wherein the biomarker is CD27.

Further embodiment 72. The use of further embodiments 66 or 67, wherein the biomarker is APP.

Further embodiment 73. The use of further embodiments 66 or 67, wherein the biomarker is the combination of CD27 and APP.

Whereas particular embodiments have been described above for purposes of illustration, it will be appreciated by those skilled in the art that numerous variations of the details may be made without departing from the invention as described in the appended claims.

1. A method of treating an autoimmune disease or reducing symptoms thereof in a human subject in need thereof, comprising:
determining an increased expression level of one or more biomarkers selected from CD27, TCF7, CD40LG, FOXP3, CD28, APP, IL-3RA (CD123), MAPIA, TRABD2A, ST6GALI, ATAD5, ATP13A2, SLC17A9, TBC1D4, MAL, ACY3, DNPHI, CNDP2, CLCN5, CALR, ST3GAL5, and USP21 in a blood sample of the human subject: and
if the expression of the one or more biomarkers is increased, administering a human anti-CD19 antibody comprising an Fc modification selected from S267E, L328F, and a combination thereof as compared to a parent IgG Fc region, wherein the numbering is according to the EU index as in Kabat.

2-3. (canceled)

4. A method of selecting one or more human subjects for treating an autoimmune disease or reducing symptoms thereof, comprising:
determining increased expression of one or more biomarkers selected from CD27, TCF7, CD40LG, FOXP3, CD28, APP, IL-3RA (CD123), MAPIA, TRABD2A, ST6GALI, ATAD5, ATP13A2, SLC17A9, TBC1D4, MAL, ACY3, DNPHI, CNDP2, CLCN5, CALR, ST3GAL5, and USP21 in the one or more subjects; and
to the subjects having increased expression, administering a human anti-CD19 antibody comprising an Fc modification selected from S267E, L328F, and a combination thereof as compared to a parent IgG Fc region, wherein the numbering is according to the EU index as in Kabat.

5. A method of treating SLE or reducing symptoms thereof in a human subject in need thereof, comprising:
determining an increased expression level of one or more biomarkers selected from CD27, TCF7, CD40LG, FOXP3, CD28, APP, IL-3RA (CD123), MAPIA, TRABD2A, ST6GALI, ATAD5, ATP13A2, SLC17A9, TBC1D4, MAL, ACY3, DNPHI, CNDP2, CLCN5, CALR, ST3GAL5, and USP21 in a blood sample of the human subject: and
if the expression of the one or more biomarkers is increased, administering a human anti-CD19 antibody comprising an Fc modification selected from S267E, L328F, and a combination thereof as compared to a parent IgG Fc region, wherein the numbering is according to the EU index as in Kabat.

6-8. (canceled)

9. The method of claim 1, wherein the one or more biomarkers are selected from CD27, TCF7, CD40LG, FOXP3, CD28, APP, IL-3RA (CD123), MAPIA, ST6GALI, SLC17A9, and CALR. 10-14. (canceled)

15. The method of claim 1, wherein the biomarker is the combination of CD27 and APP.

16. The method of claim 1, wherein the determining or identifying step comprises administering a genotyping test, a proteomic test, or a transcriptomic test to the blood sample of the subject.

17. (canceled)

18. The method claim 1, wherein if the expression of one or more biomarkers is not increased then the antibody is withheld from the subject.

19. The method of claim 1, wherein the blood sample is whole blood.

20. The method of claim 1, wherein the blood sample is selected from T cells, plasmablasts, and a combination thereof.

21. The method of claim 1, wherein the blood sample comprises plasmacytoid dendritic cells.

22. The method of claim 1, wherein the antibody comprises:
a light chain comprising a variable region having a CDRI comprising SEQ ID NO: 10, a CDR2 comprising SEQ ID NO: 11, and a CDR3 comprising SEQ ID NO: 12:
a heavy chain comprising a variable region having a CDRI comprising SEQ ID NO: 13, a CDR2 comprising SEQ ID NO: 14, and a CDR3 comprising SEQ ID NO: 15, and as compared to SEQ ID NO: 4; and
the Fc modification is S267E and L328F as compared to SEQ ID NO: 4,
wherein the numbering is according to the EU index as in Kabat.

23. The method of claim 1, wherein the antibody comprises
a light chain; and
a heavy chain comprising an amino acid sequence of SEQ ID NO: 2 and amino acid substitutions in the Fc region S267E and L328F as compared to SEQ ID NO: 4, wherein the numbering is according to the EU index as in Kabat.

24. The method of claim 1, wherein the antibody comprises
a light chain comprising an amino acid sequence of SEQ ID NO: 7: and
a heavy chain comprising an amino acid sequence of SEQ ID NO: 9.

25. The method of claim 1, wherein the severity of the disease within said subject is reduced and/or the days to loss of improvement (LOI) is increased.

26. The method of claim 1, wherein the human anti-CD19 antibody is administered subcutaneously.

27. The method of claim 1, further comprising obtaining the blood sample from the subject.

28. The method of claim 1, wherein the autoimmune disease is selected from SLE and rheumatoid arthritis.

29-47. (canceled)

48. An in vitro method of improving therapeutic efficacy for treatment of an autoimmune disease, comprising:
determining the expression of one or more biomarkers selected from CD27, TCF7, CD40LG, FOXP3, CD28, APP, IL-3RA (CD123), MAPIA, TRABD2A, ST6GALI, ATAD5, ATP13A2, SLC17A9, TBCID4, MAL, ACY3, DNPHI, CNDP2, CLCN5, CALR, ST3GAL5, and USP21 in a sample from a subject having the autoimmune disease,
wherein an increase of the expression of the one or more biomarkers indicates the efficacy of a human anti-CD19 antibody in the subject.

49. An in vitro method of determining susceptibility to treatment for an autoimmune disease in a human subject in need thereof, comprising:
determining the expression of one or more biomarkers selected from CD27, TCF7, CD40LG, FOXP3, CD28, APP, IL-3RA (CD123), MAPIA, TRABD2A, ST6GALI, ATAD5, ATP13A2, SLC17A9, TBCID4, MAL, ACY3, DNPHI, CNDP2, CLCN5, CALR, ST3GAL5, and USP21 in a sample from the subject, wherein the subject has been treated with a human anti-CD19 antibody,
wherein an increase of the expression of the one or more biomarkers indicates the efficacy of a human anti-CD19 antibody in the subject.

50-73. (canceled)
